(19) United States
(12) Patent Application Publication
(10) Pub. No.: US 2009/0186414 A1
(43) Pub. Date: Jul. 23, 2009

(54) Methods of Generating Cardiomyocytes and Cardiac Progenitors and Compositions

(76) Inventors: DEEPAK SRIVASTAVA, SAN FRANCISCO, CA (US); KATHRYN N. IVEY, SAN FRANCISCO, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/355,519

(22) Filed: Jan. 16, 2009

Related U.S. Application Data
(60) Provisional application No. 61/022,081, filed on Jan. 18, 2008.

(51) Int. Cl. C12N 15/00; C12N 5/06

(52) U.S. Cl. 435/455; 435/325

(57) ABSTRACT

The present disclosure provides methods of inducing cardiomyogenesis in a stem cell or progenitor cell, or in a population of stem cells or progenitor cells; and methods for expansion of (increasing the numbers of) cardiac progenitors. Cell compositions are also provided.

### CROSS-REFERENCE

This application claims the benefit of U.S. Provisional Patent Application No. 61/022,081, filed Jan. 18, 2008, which application is incorporated herein by reference in its entirety.

### BACKGROUND

Embryonic stem (ES) cells, derived from the inner cell mass of blastocysts, are pluripotent and self-renewing cells, with the ability to give rise to all three germ layers-ectoderm, mesoderm, and endoderm. Numerous signaling pathways, including those involving members of the Wnt, Bmp, and Notch pathways, appear to regulate cell fate during embryogenesis and can be utilized in various forms to influence lineage choices in cultured ES cells. Such pathways often culminate in transcriptional events, either through DNA-binding proteins or chromatin remodeling factors, which dictate which subset of the genome is activated or silenced in specific cell types. As a result, transcription factors that regulate pluripotency or lineage-specific gene and protein expression have been a major focus of ES cell research.

In addition to transcriptional regulation, post-transcriptional control by small noncoding RNAs such as microRNAs (miRNAs) quantitatively influences the ultimate proteome. miRNAs are naturally occurring RNAs that are transcribed in the nucleus, often under the control of specific enhancers, and are processed by the RNAses DroshaIDGCR8 and Dicer into mature ˜22 nucleotide RNAs that bind to complementary targets in RNAs. miRNA:mRNA interactions in RNA-induced silencing complexes can result in mRNA degradation, deadenylation, or translational repression at the level of the ribosome. Over 450 human miRNAs have been described, and each is predicted to target tens if not hundreds of different mRNAs. Because they can regulate numerous genes, often in common pathways, miRNAs are candidates for master regulators of cellular processes, much like transcription factors that regulate entire programs of cellular differentiation and organogenesis.

During differentiation of ES cells into aggregates called embryoid bodies (EBs), which to a limited extent recapitulate embryonic development, cardiomyocytes are among the first cell types to arise. They become easily visible 7 days after differentiation as small clusters of rhythmically and synchronously contracting cells. Like naturally occurring cardiac muscle cells, ES cell-derived cardiomyocytes express markers of cardiac differentiation, assemble contractile machinery, and establish cell-cell communication.

### Literature

Zhao et al. (2007) Cell 129:303; Zhao and Srivastava (2007) Trends Biochem. Sci. 32:189; Kwon et al. (2005) Proc. Natl. Acad. Sci. USA 102:18986; Nguyen and Frasch (2006) Curr. Opin. Genet. Dev. 16:533; Ivey et al. (Jan. 22, 2007) Keystone Symposium: Molecular Pathways in Cardiac Development and Disease Abstract: “MicroRNAs regulate cardiomyocyte differentiation from embryonic stem cells.”

### SUMMARY OF THE INVENTION

The present disclosure provides methods of inducing cardiomyogenesis in a stem cell or progenitor cell, or in a population of stem cells or progenitor cells; and methods for expansion of (increasing the numbers of) cardiac progenitors. Cell compositions are also provided.

### BRIEF DESCRIPTION OF THE DRAWINGS

FIGS. 1A-C depict identification of miRNAs expressed in ES cell-derived cardiomyocytes.

FIGS. 2A-I depict the effects of miR-1 and miR-133 on mesoderm differentiation.

FIGS. 3A-F depict the effect of miR-1 and miR-133 on endoderm and neuroectoderm differentiation in mES cells.

FIGS. 4A-D depict results showing that Dll-1 protein levels are negatively regulated by miR-1 in mES cells, and that knockdown of Dll-1 expression recapitulates many effects of miR-1 expression.

FIGS. 5A-C depict the effects of miR-1 or miR-133 expression in hES cells.

FIG. 6 depicts an alignment of miR-1 nucleotide sequences.

FIG. 7 depicts an alignment of miR-133a-1 and miR-133a-2 nucleotide sequences.

FIG. 8 depicts an alignment of miR-133b nucleotide sequences.

### DEFINITIONS

As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA sequences have been described in publications such as, Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179, which are incorporated herein by reference. Examples of microRNAs include any RNA that is a fragment of a larger RNA or is a miRNA, siRNA, stRNA, sncRNA, tncRNA, snoRNA, smRNA, snRNA, or other small non-coding RNA. See, e.g., US Patent Applications 20050272923, 20050266552, 20050142581, and 20050075492. A “microRNA precursor” refers to a nucleic acid having a stem-loop structure with a microRNA sequence incorporated therein.

A “stem-loop structure” refers to a nucleic acid having a secondary structure that includes a region of nucleotides which are known or predicted to form a double strand (step portion) that is linked on one side by a region of predominantly single-stranded nucleotides (loop portion). The terms “hairpin” and “fold-back” structures are also used herein to refer to stem-loop structures. Such structures are well known in the art and these terms are used consistently with their known meanings in the art. The actual primary sequence of nucleotides within the stem-loop structure is not critical to the practice of the invention as long as the secondary structure is present. As is known in the art, the secondary structure does not require exact base-pairing. Thus, the stem may include one or more base mismatches. Alternatively, the base-pairing may be exact, i.e. not include any mismatches.

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, fetal, post-natal, juvenile or adult tissue. The term “progenitor cell”, as used herein, refers to an undifferentiated cell derived from a stem cell, and is not itself a stem cell. Some progenitor cells can produce progeny that are capable of differentiating into more than one cell type.

The term “induced pluripotent stem cell” (or “iPS cell”), as used herein, refers to a stem cell induced from a somatic cell, e.g., a differentiated somatic cell, and that has a higher potency than said somatic cell. iPS cells are capable of self-renewal and differentiation into mature cells, e.g. cells of mesodermal lineage or cardiomyocytes. iPS may also be capable of differentiation into cardiac progenitor cells.

As used herein the term “isolated” with reference to a cell, refers to a cell that is in an environment different from that in which the cell naturally occurs, e.g., where the cell naturally occurs in a multicellular organism, and the cell is removed from the multicellular organism, the cell is “isolated.” An isolated genetically modified host cell can be present in a mixed population of genetically modified host cells, or in a mixed population comprising genetically modified host cells and host cells that are not genetically modified. For example, an isolated genetically modified host cell can be present in a mixed population of genetically modified host cells in vitro, or in a mixed in vitro population comprising genetically modified host cells and host cells that are not genetically modified.

A “host cell,” as used herein, denotes an in vivo or in vitro cell (e.g., a eukaryotic cell cultured as a unicellular entity), which eukaryotic cell can be, or has been, used as recipients for a nucleic acid (e.g., an exogenous nucleic acid), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation.

The term “genetic modification” and refers to a permanent or transient genetic change induced in a cell following introduction of new nucleic acid (i.e., nucleic acid exogenous to the cell). Genetic change (“modification”) can be accomplished by incorporation of the new nucleic acid into the genome of the host cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

As used herein, the term “exogenous nucleic acid” refers to a nucleic acid that is not normally or naturally found in and/or produced by a cell in nature, and/or that is introduced into the cell (e.g., by electroporation, transfection, infection, lipofection, or any other means of introducing a nucleic acid into a cell).

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

A “therapeutically effective amount” or “efficacious amount” means the amount of a compound or a number of cells that, when administered to a mammal or other subject for treating a disease, is sufficient to effect such treatment for the disease. The “therapeutically effective amount” will vary depending on the compound or the cell, the disease and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a microRNA” (or “a miRNA”) includes a plurality of such microRNAs (miRNAs) and reference to “the stem cell” includes reference to one or more stem cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of inducing cardiomyogenesis in a stem cell or progenitor cell, or in a population of stem cells or progenitor cells. The methods generally involve introducing into a stem cell or progenitor cell a microRNA (miRNA) that specifically targets one or more mRNAs and, as a consequence of said targeting, induces differentiation of the stem cell or progenitor cell. The present disclosure further provides methods for expansion of (increasing the numbers of) cardiac progenitors. The methods generally involve introducing into a stem cell or progenitor cell a miRNA that specifically targets one or more mRNAs and, as a consequence of said targeting, induces proliferation of cardiac progenitors. The present disclosure further provides compositions comprising genetically modified stem cells and/or genetically modified progenitor cells. The present disclosure also provides compositions of cells (e.g., cardiomyocytes, cardiac progenitor cells) generated from the methods described herein.

In some embodiments, a subject method provides for differentiation of a stem cell or progenitor cell, or a population of stem cells or progenitor cells, into a cardiomyocyte(s). In other words, in some embodiments, a subject method provides for induction of cardiomyogenesis in a stem cell or a progenitor cell. In some of these embodiments, a subject method involves introducing into a stem or progenitor cell a miR-1 nucleic acid, or a nucleic acid comprising a nucleotide sequence encoding a miR-1 nucleic acid. In other embodiments, a subject method involves introducing into a stem or progenitor cell a miR-133 nucleic acid, or a nucleic acid comprising a nucleotide sequence encoding a miR-133 nucleic acid. In other embodiments, a subject method involves introducing into a stem or progenitor cell a miR-1 nucleic acid and a miR-133 nucleic acid, or a nucleic acid(s) comprising nucleotide sequences encoding a miR-1 nucleic acid and a miR-133 nucleic acid. In some embodiments, a suitable miR-1 or miR-133 nucleic acid comprises a stem-loop forming (“precursor”) nucleotide sequence. In other embodiments, a suitable miR-1 or miR-133 nucleic acid comprises a mature form of a miR-1 or a miR-133 nucleic acid.

In some embodiments, introduction of a miR-1 nucleic acid, or a miR-1-encoding nucleic acid into a stem cell or progenitor cell (e.g., a cardiac progenitor cell) targets a Notch ligand Delta-like-1 (Dll-1) nucleic acid in the cell. For example, a miR-1 nucleic acid can target a Dll-1 nucleic acid comprising a nucleotide sequence having at least about 70%, at least about 75%, at least about 80%, at least about 85%, at least about 90%, at least about 95%, at least about 98%, at least about 99%, or 100%, nucleotide sequence identity to the nucleotide sequence set forth in SEQ ID NO:9 (a Homo sapiens Dll-1 nucleotide sequence), or the complement thereof.

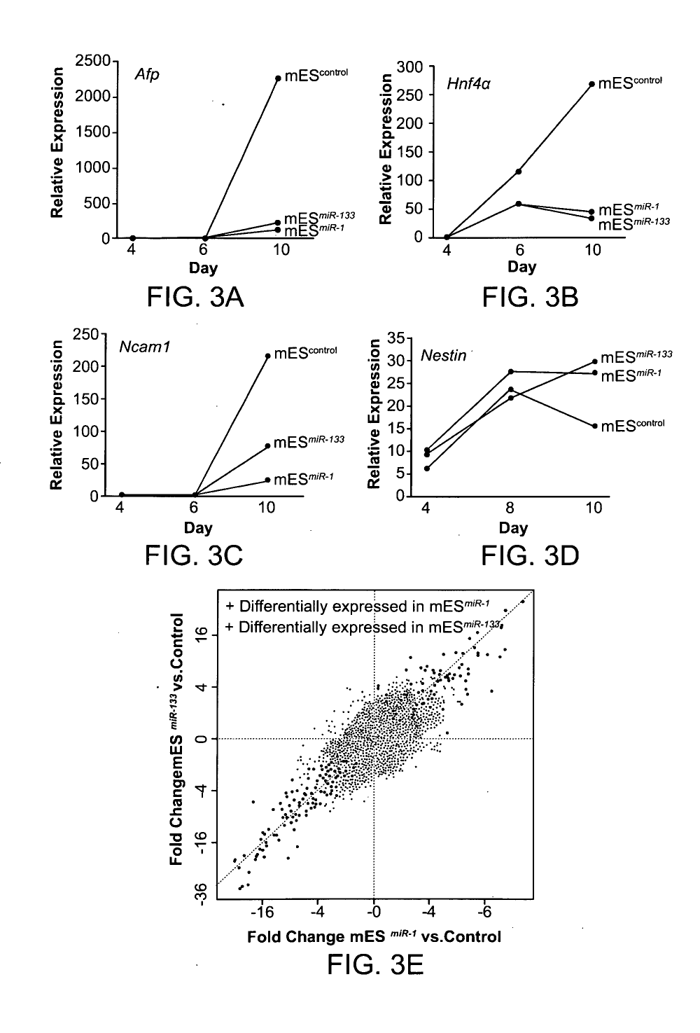

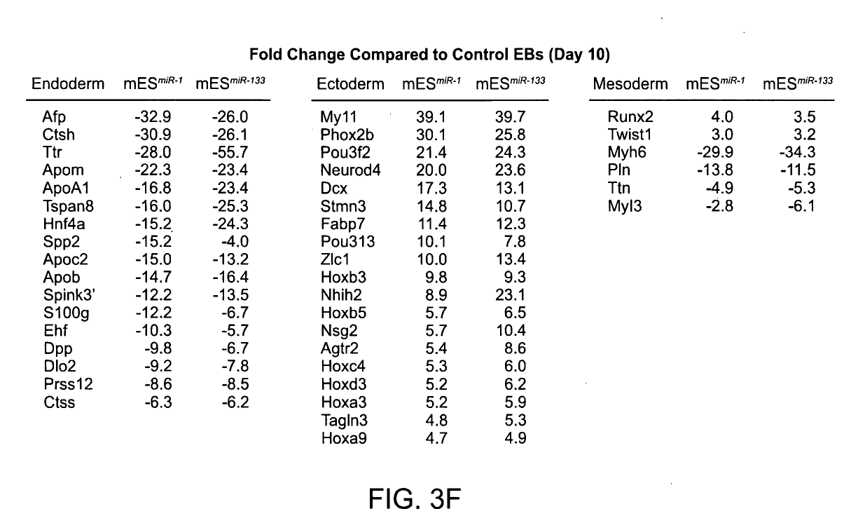

In some embodiments, introduction of a miR-1 nucleic acid, or a miR-1-encoding nucleic acid into a stem cell or progenitor cell (e.g., a cardiac progenitor cell) results in reduced expression of one or more endoderm-specific genes, e.g., introduction of a miR-1 nucleic acid, or a miR-1-encoding nucleic acid into a stem cell or progenitor cell (e.g., a cardiac progenitor cell) results in reduced expression of one or more of Afp, Ctsh, Ttr, Apom, ApoA1, Tspan8, Hnf4a, Spp2, Apoc2, Apob, Spink3, S100g, Ehf, Dpp, Dlo1, Prss12, and Ctss, as shown in FIG. 3F. Introduction of a miR-1 nucleic acid, or a miR-1-encoding nucleic acid into a stem cell or progenitor cell (e.g., a cardiac progenitor cell) results in a reduction of from about 5-fold to about 10-fold, from about 10-fold to about 15-fold, from about 15-fold to about 20-fold, from about 20-fold to about 25-fold, from about 20-fold to about 25-fold, or from about 25-fold to about 30-fold, in the expression level (e.g., mRNA level) of one or more of Afp, Ctsh, Ttr, Apom, ApoA1, Tspan8, Hnf4a, Spp2, Apoc2, Apob, Spink3, S100g, Ehf, Dpp, Dlo1, Prss12, and Ctss.

In some embodiments, introduction of a miR-133 nucleic acid, or a miR-133-encoding nucleic acid into a stem cell or progenitor cell (e.g., a cardiac progenitor cell) targets a Notch ligand Delta-like-1 (Dll-1) nucleic acid. For example, a miR-133 nucleic acid can target a Dll-1 nucleic acid comprising a nucleotide sequence having at least about 70%, at least about 75%, at least about 80%, at least about 85%, at least about 90%, at least about 95%, at least about 98%, at least about 99%, or 100%, nucleotide sequence identity to the nucleotide sequence set forth in SEQ ID NO:9 (a Homo sapiens Dll-1 nucleotide sequence), or the complement thereof.

In some embodiments, introduction of a miR-133 nucleic acid, or a miR-133-encoding nucleic acid into a stem cell or progenitor cell (e.g., a cardiac progenitor cell) results in reduced expression of one or more endoderm-specific genes, e.g., introduction of a miR-133 nucleic acid, or a miR-133-encoding nucleic acid into a stem cell or progenitor cell (e.g., a cardiac progenitor cell) results in reduced expression of one or more of Afp, Ctsh, Ttr, Apom, ApoA1, Tspan8, Hnf4a, Spp2, Apoc2, Apob, Spink3, S100g, Ehf, Dpp, Dlo1, Prss12, and Ctss, as shown in FIG. 3F. Introduction of a miR-133 nucleic acid, or a miR-133-encoding nucleic acid into a stem cell or progenitor cell (e.g., a cardiac progenitor cell) results in a reduction of from about 5-fold to about 10-fold, from about 10-fold to about 15-fold, from about 15-fold to about 20-fold, from about 20-fold to about 25-fold, from about 20-fold to about 25-fold, or from about 25-fold to about 30-fold, in the expression level (e.g., mRNA level) of one or more of Afp, Ctsh, Ttr, Apom, ApoA1, Tspan8, Hnf4a, Spp2, Apoc2, Apob, Spink3, S100g, Ehf, Dpp, Dlo1, Prss12, and Ctss.

In some embodiments, introduction of a miR-1 nucleic acid, or a miR-1-encoding nucleic acid into a stem cell or progenitor cell (e.g., a cardiac progenitor cell) results in increased expression of one or more ectoderm-specific genes (e.g., markers associated with neuroectoderm specification or early neural differentiation), e.g., introduction of a miR-1 nucleic acid, or a miR-1-encoding nucleic acid into a stem cell or progenitor cell (e.g., a cardiac progenitor cell) results in increased expression of one or more of Myt1, Phox2b, Pou3f2, Neurod4, Dcx, Stmn3, Fabp7, Pou3f3, Zic1, Hoxb3, Nhlh2, Hoxb5, Nsg2, Agtr2, Hoxc4, Hoxd3, Hoxa3, Tagln3, and Hoxa9, as shown in FIG. 3F. Introduction of a miR-1 nucleic acid, or a miR-1-encoding nucleic acid into a stem cell or progenitor cell (e.g., a cardiac progenitor cell) results in an increase of from about 4-fold to about 5-fold, from about 5-fold to about 10-fold, from about 10-fold to about 15-fold, from about 15-fold to about 20-fold, from about 20-fold to about 25-fold, from about 25-fold to about 30-fold, from about 30-fold to about 35-fold, or from about 35-fold to about 40-fold, in the expression level (e.g., mRNA level) of one or more of: Myt1, Phox2b, Pou3f2, Neurod4, Dcx, Stmn3, Fabp7, Pou3f3, Zic1, Hoxb3, Nhlh2, Hoxb5, Nsg2, Agtr2, Hoxc4, Hoxd3, Hoxa3, Tagln3, and Hoxa9.

In some embodiments, introduction of a miR-133 nucleic acid, or a miR-133-encoding nucleic acid into a stem cell or progenitor cell (e.g., a cardiac progenitor cell) results in increased expression of one or more ectoderm-specific genes (e.g., markers associated with neuroectoderm specification or early neural differentiation), e.g., introduction of a miR-133 nucleic acid, or a miR-133-encoding nucleic acid into a stem cell or progenitor cell (e.g., a cardiac progenitor cell) results in increased expression of one or more of Myt1, Phox2b, Pou3f2, Neurod4, Dcx, Stmn3, Fabp7, Pou3f3, Zic1, Hoxb3, Nhlh2, Hoxb5, Nsg2, Agtr2, Hoxc4, Hoxd3, Hoxa3, Tagln3, and Hoxa9, as shown in FIG. 3F. Introduction of a miR-133 nucleic acid, or a miR-133-encoding nucleic acid into a stem cell or progenitor cell (e.g., a cardiac progenitor cell) results in an increase of from about 4-fold to about 5-fold, from about 5-fold to about 10-fold, from about 10-fold to about 15-fold, from about 15-fold to about 20-fold, from about 20-fold to about 25-fold, from about 25-fold to about 30-fold, from about 30-fold to about 35-fold, or from about 35-fold to about 40-fold, in the expression level (e.g., mRNA level) of one or more of: Myt1, Phox2b, Pou3f2, Neurod4, Dcx, Stmn3, Fabp7, Pou3f3, Zic1, Hoxb3, Nhlh2, Hoxb5, Nsg2, Agtr2, Hoxc4, Hoxd3, Hoxa3, Tagln3, and Hoxa9.

In some embodiments, introduction of a miR-1 nucleic acid or a miR-1-encoding nucleic acid into a stem cell or progenitor cell (e.g., a cardiac progenitor cell) results in differentiation of the stem cell or progenitor cell into a cardiomyocyte. A cardiomyocyte will generally express on its cell surface and/or in the cytoplasm one or more cardiac-specific markers. Suitable cardiomyocyte-specific markers include, but are not limited to, cardiac troponin I, cardiac troponin-C, tropomyosin, caveolin-3, GATA-4, myosin heavy chain, myosin light chain-2a, myosin light chain-2v, ryanodine receptor, sarcomeric α-actinin, NRx2.5, MEF-2c, and atrial natriuretic factor. In some embodiments, introduction of a miR-1 nucleic acid or a miR-1-encoding nucleic acid into a stem cell or progenitor cell (e.g., a cardiac progenitor cell) results in generation of a cardiomyocyte that expresses one or more cardiac-specific markers. In some embodiments, introduction of a miR-1 nucleic acid or a miR-1-encoding nucleic acid into a stem cell or progenitor cell (e.g., a cardiac progenitor cell) results in generation of beating cardiomyocytes. The expression of various markers specific to cardiomyocytes is detected by conventional biochemical or immunochemical methods (e.g., enzyme-linked immunosorbent assay; immunohistochemical assay; and the like). Alternatively, expression of nucleic acid encoding a cardiomyocyte-specific marker can be assessed. Expression of cardiomyocyte-specific marker-encoding nucleic acids in a cell can be confirmed by reverse transcriptase polymerase chain reaction (RT-PCR) or hybridization analysis, molecular biological methods which have been commonly used in the past for amplifying, detecting and analyzing mRNA coding for any marker proteins. Nucleic acid sequences coding for markers specific to cardiomyocytes are known and are available through public data bases such as GenBank; thus, marker-specific sequences needed for use as primers or probes is easily determined.

In some embodiments, introduction of a miR-133 nucleic acid or a miR-133-encoding nucleic acid into a stem cell or progenitor cell (e.g., a cardiac progenitor cell) results in an increase in the number of cardiac progenitor cells. For example, introduction of a miR-133 nucleic acid or a miR-133-encoding nucleic acid into a stem cell or cardiac progenitor cell results in an increase of from about 2-fold to about 5-fold, from about 5-fold to about 10-fold, from about 10-fold to about 25-fold, from about 25-fold to about 50-fold, from about 50-fold to about 100-fold, from about 10²-fold to about 5×10²-fold, from about 5×10²-fold to about 10³-fold, from about 10³-fold to about 10⁴-fold, or greater than 10⁴-fold.

In some embodiments, a miR-1 and/or a miR-133 nucleic acid (or a nucleic acid comprising a nucleotide sequence encoding miR-1 and/or miR-133) is introduced into a population of cells that comprises stem cells and/or cardiac progenitor cells; and, as a result, the proportion of cells in the population that are cardiomyocytes or cardiac progenitor cells increases. For example, in some embodiments, introduction of a miR-1 nucleic acid, or a nucleic acid comprising a nucleotide sequence encoding miR-1, into a cell population that comprises stem cells or cardiac progenitor cells results in differentiation of at least about 10% of the stem cell or progenitor cell population into cardiomyocytes. For example, in some embodiments, from about 10% to about 50% of the stem cell or progenitor cell population differentiates into cardiomyocytes. In other embodiments, at least about 50% of the stem cell or progenitor cell population differentiates into cardiomyocytes. For example, in some embodiments, from about 50% to about 60%, from about 60% to about 70%, from about 70% to about 80%, or from about 80% to about 90%, or more, of the stem cell or progenitor cell population differentiates into cardiomyocytes.

In some embodiments, a subject method involves: a) introducing into a stem cell a miR-133 nucleic acid, or a miR-133-encoding nucleic acid, thereby increasing the number of cardiac progenitor cells; and b) introducing into the cardiac progenitor cells a miR-1 nucleic acid or a miR-1-encoding nucleic acid, thereby inducing differentiation of the cardiac progenitor cells into cardiomyocytes.

Suitable stem cells include embryonic stem cells, adult stem cells, and induced pluripotent stem (iPS) cells.

iPS cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

iPS cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT.

Methods of generating iPS cells are known in the art, and a wide range of methods can be used to generate iPS cells. See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et. al. (2008) Science 321:1218-1221; Blelloch et al. (2007) Cell Stem Cell 1:245-247; Stadtfeld et al. (2008) Science 322:945-949; Stadtfeld et al. (2008) 2:230-240; Okita et al. (2008) Science 322:949-953.

In some embodiments, iPS cells are generated from somatic cells by forcing expression of a set of factors in order to promote increased potency of a cell or de-differentiation. Forcing expression can include introducing expression vectors encoding polypeptides of interest into cells, introducing exogenous purified polypeptides of interest into cells, or contacting cells with a reagent that induces expression of an endogenous gene encoding a polypeptide of interest.

Forcing expression may include introducing expression vectors into somatic cells via use of moloney-based retroviruses (e.g., MLV), lentiviruses (e.g., HIV), adenoviruses, protein transduction, transient transfection, or protein transduction. In some embodiments, the moloney-based retroviruses or HIV-based lentiviruses are pseudotyped with envelope from another virus, e.g. vesicular stomatitis virus g (VSV-g) using known methods in the art. See, e.g. Dimos et al. (2008) Science 321:1218-1221.

In some embodiments, iPS cells are generated from somatic cells by forcing expression of Oct-3/4 and Sox2 polypeptides. In some embodiments, iPS cells are generated from somatic cells by forcing expression of Oct-3/4, Sox2 and Klf4 polypeptides. In some embodiments, iPS cells are generated from somatic cells by forcing expression of Oct-3/4, Sox2, Klf4 and c-Myc polypeptides. In some embodiments, iPS cells are generated from somatic cells by forcing expression of Oct-4, Sox2, Nanog, and LIN28 polypeptides.

For example, iPS cells can be generated from somatic cells by genetically modifying the somatic cells with one or more expression constructs encoding Oct-3/4 and Sox2. As another example, iPS cells can be generated from somatic cells by genetically modifying the somatic cells with one or more expression constructs comprising nucleotide sequences encoding Oct-3/4, Sox2, c-myc, and Klf4. As another example, iPS cells can be generated from somatic cells by genetically modifying the somatic cells with one or more expression constructs comprising nucleotide sequences encoding Oct-4, Sox2, Nanog, and LIN28.

In some embodiments, cells undergoing induction of pluripotency as described above, to generate iPS cells, are contacted with additional factors which can be added to the culture system, e.g., included as additives in the culture medium. Examples of such additional factors include, but are not limited to: histone deacetylase (HDAC) inhibitors, see, e.g. Huangfu et al. (2008) Nature Biotechnol. 26:795-797; Huangfu et al. (2008) Nature Biotechnol. 26: 1269-1275; DNA demethylating agents, see, e.g., Mikkelson et al (2008) Nature 454, 49-55; histone methyltransferase inhibitors, see, e.g., Shi et al. (2008) Cell Stem Cell 2:525-528; L-type calcium channel agonists, see, e.g., Shi et al. (2008) 3:568-574; Wnt3a, see, e.g., Marson et al. (2008) Cell 134:521-533; and siRNA, see, e.g., Zhao et al. (2008) Cell Stem Cell 3: 475-479.

In some embodiments, iPS cells are generated from somatic cells by forcing expression of Oct3/4, Sox2 and contacting the cells with an HDAC inhibitor, e.g., valproic acid. See, e.g., Huangfu et al. (2008) Nature Biotechnol. 26: 1269-1275. In some embodiments, iPS cells are generated from somatic cells by forcing expression of Oct3/4, Sox2, and Klf4 and contacting the cells with an HDAC inhibitor, e.g., valproic acid. See, e.g., Huangfu et al. (2008) Nature Biotechnol. 26:795-797.

In some embodiments, a subject method comprises: a) inducing a somatic cell from an individual to become a pluripotent stem cell, generating an iPS cell; b) introducing a miR-1 nucleic acid (or a nucleic acid comprising a nucleotide sequence encoding miR-1) into the iPS cell, generating cardiomyocytes. Such cardiomyocytes would be useful for introducing into the individual from whom the somatic cell was obtained. Such cardiomyocytes could also be introduced into an individual other than the individual from whom the somatic cell was obtained. For example, in some embodiments, a somatic cell is obtained from a donor individual; an iPS cell is generated from the somatic cell; the iPS cell is induced to differentiate into a cardiomyocyte; and the cardiomyocyte is introduced into a recipient individual, where the recipient individual is not the same individual as the donor individual.

In other embodiments, a subject method comprises: a) inducing a somatic cell from an individual to become a pluripotent stem cell, generating an iPS cell; b) introducing a miR-133 nucleic acid (or a nucleic acid comprising a nucleotide sequence encoding miR-133) into the iPS cell, generating cardiac progenitor cells. Such cardiac progenitor cells would be useful for introducing into the individual from whom the somatic cell was obtained. Such cardiac progenitor cells could also be introduced into an individual other than the individual from whom the somatic cell was obtained. For example, in some embodiments, a somatic cell is obtained from a donor individual; an iPS cell is generated from the somatic cell; the iPS cell is induced to differentiate into a cardiac progenitor cell; and the cardiac progenitor cell is introduced into a recipient individual, where the recipient individual is not the same individual as the donor individual.

In some embodiments, a subject method comprises: a) inducing a somatic cell from a donor individual to become a pluripotent stem cell, generating an iPS cell; b) introducing a miR-133 (or a nucleic acid comprising a nucleotide sequence encoding miR-133) into the iPS cell, generating cardiac progenitor cells; and c) introducing a miR-1 nucleic acid (or a nucleic acid comprising a nucleotide sequence encoding miR-1) into the cardiac progenitor cells, thereby generating cardiomyocytes. In some embodiments, the cardiomyocytes thus generated are introduced back into the donor individual. In other embodiments, the cardiomyocytes thus generated are introduced into a recipient individual, where the recipient individual is not the same individual as the donor individual.

miR-1 Nucleic Acids

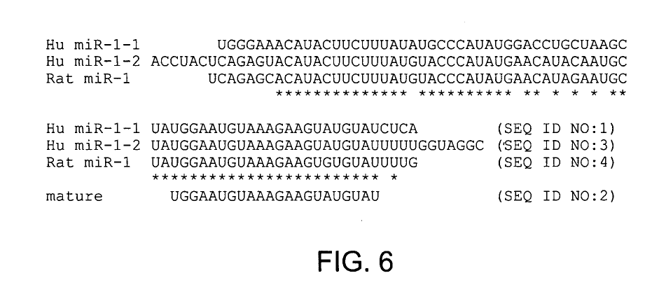

In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:1 and depicted in FIG. 6. In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:3 and depicted in FIG. 6. In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:4 and depicted in FIG. 6.

In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 7 to 69 of the nucleotide sequence set forth in SEQ ID NO:1 and depicted in FIG. 6. In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 14-76 of the nucleotide sequence set forth in SEQ ID NO:3 and depicted in FIG. 6. In some embodiments, a suitable miR-1 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 8 to 70 of the nucleotide sequence set forth in SEQ ID NO:4 and depicted in FIG. 6.

Other suitable miR-1 nucleic acids include a nucleic acid comprising a nucleotide sequence having at least about 75%, at least about 80%, at least about 85%, at least about 90%, at least about 95%, at least about 98%, at least about 99%, or 100%, nucleotide sequence identity to one or more of: a rat miR-1 nucleotide sequence (see, e.g., GenBank Accession No. DQ066650; and Zhao et al. (2005) Nature 436:214); a frog miR-1 nucleotide sequence (see, e.g., GenBank Accession No. DQ066652); and a zebrafish miR-1 nucleotide sequence (see, e.g., GenBank Accession No. DQ066651).

In some embodiments, a suitable miR-1 nucleic acid comprises the nucleotide sequence 5′-UGGAAUGUAAAGAAGUAUGUAU-3′ (SEQ ID NO:2), or a nucleotide sequence that has at least about 80%, at least about 85%, at least about 90%, at least about 95%, at least about 98%, or at least about 99%, nucleotide sequence identity over the 22-nucleotide sequence of SEQ ID NO:2. In some embodiments, a suitable miR-1 nucleic acid has a length of 22 nucleotides. In other embodiments, a suitable miR-1 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:2, and has one or more additional nucleotides 5′- and/or 3′ of the 22-nucleotide core sequence. Thus, e.g., in some embodiments, a suitable miR-1 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:2, and has a length of from about 23 nucleotides to about 25 nucleotides, from about 25 nucleotides to about 30 nucleotides, from about 30 nucleotide to about 50 nucleotides, from about 50 nucleotides to about 100 nucleotides, from about 0.1 kb to about 0.5 kb, from about 0.5 kb to about 1 kb, from about 1 kb to about 1.5 kb, from about 1.5 kb to about 2 kb, from about 2 kb to about 3 kb, from about 3 kb to about 5 kb, from about 5 kb to about 10 kb, or greater than 10 kb.

In some embodiments, a suitable miR-1 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:2, and further includes a nucleotide sequence that is complementary to the 22-nucleotide core sequence. The complementary sequence will have a length of from about 18 nucleotides to about 26 nucleotides, and will have a nucleotide sequence that has from 80% to 85%, from 85% to 90%, from 90% to 95%, 96%, 97%, 98%, 99%, or 100%, nucleotide sequence identity to the 22-nucleotide core sequence. The 22-nucleotide core sequence and the complementary sequence are separated from one another by 1 nucleotide, 2 nucleotides (nt), 3 nt, 4 nt, 5 nt, 6 nt, 7 nt, 8 nt, 9 nt, 10 nt, 11 nt, 12 nt, 13 nt, 14 nt, 15 nt, 15-20 nt, 20-25 nt, or more than 25 nt.

A suitable miR-1-encoding nucleic acid comprises a nucleotide sequence encoding a miR-1 nucleic acid as described above. In some embodiments, an miR-1-encoding nucleic acid is contained within an expression vector. In some embodiments, a nucleotide sequence encoding an miR-1 nucleic acid is operably linked to a transcriptional regulatory element, e.g., a promoter, an enhancer, etc.

miR-133 Nucleic Acids

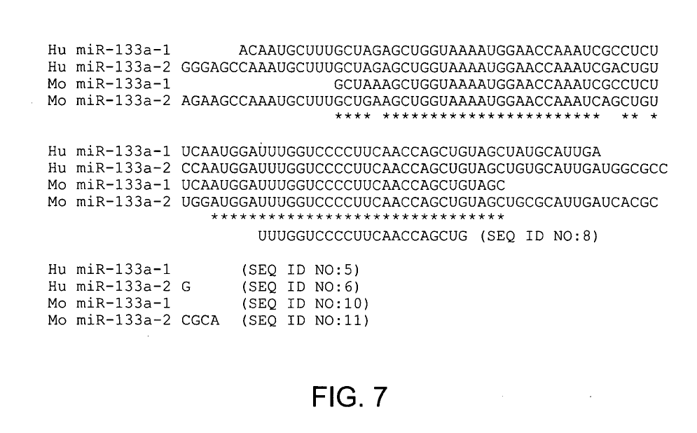

In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:5 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:6 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:10 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:11 and depicted in FIG. 7.

In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 11-78 of the nucleotide sequence set forth in SEQ ID NO:5 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 17-84 of the nucleotide sequence set forth in SEQ ID NO:6 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 1-68 of the nucleotide sequence set forth in SEQ ID NO:10 and depicted in FIG. 7. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to nucleotides 17-84 of the nucleotide sequence set forth in SEQ ID NO:11 and depicted in FIG. 7.

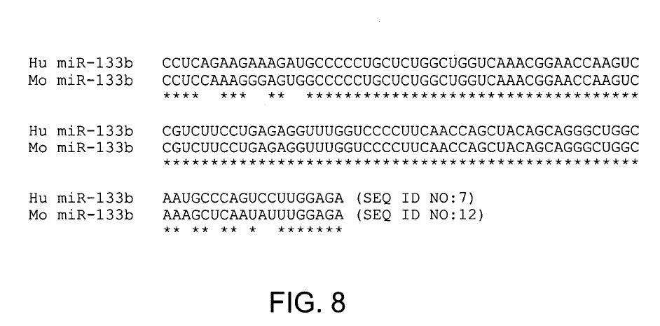

In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:7 and depicted in FIG. 8. In some embodiments, a suitable miR-133 nucleic acid comprises a nucleotide sequence having at least about 75%, at least about 80%, at least about 85%, at least about 90%, at least about 95%, at least about 98%, at least about 99%, or 100%, nucleotide sequence identity to the nucleotide sequence set forth in SEQ ID NO:12 and depicted in FIG. 8.

In some embodiments, a suitable miR-133 nucleic acid comprises the nucleotide sequence 5′-UUUGGUCCCCUUCAACCAGCUG-3′ (SEQ ID NO:8), or a nucleotide sequence that has at least about 80%, at least about 85%, at least about 90%, at least about 95%, at least about 98%, or at least about 99%, nucleotide sequence identity over the 22-nucleotide sequence of SEQ ID NO:8. In some embodiments, a suitable miR-133 nucleic acid has a length of 22 nucleotides. In other embodiments, a suitable miR-133 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:8, and has one or more additional nucleotides 5′- and/or 3′ of the 22-nucleotide core sequence. Thus, e.g., in some embodiments, a suitable miR-133 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:8, and has a length of from about 23 nucleotides to about 25 nucleotides, from about 25 nucleotides to about 30 nucleotides, from about 30 nucleotide to about 50 nucleotides, from about 50 nucleotides to about 100 nucleotides, from about 0.1 kb to about 0.5 kb, from about 0.5 kb to about 1 kb, from about 1 kb to about 1.5 kb, from about 1.5 kb to about 2 kb, from about 2 kb to about 3 kb, from about 3 kb to about 5 kb, from about 5 kb to about 10 kb, or greater than 10 kb.

In some embodiments, a suitable miR-133 nucleic acid comprises a 22-nucleotide core sequence having at least about 80%, at least about 85%, at least about 90%, at least about 95%, at least about 98%, at least about 99%, or 100%, nucleotide sequence identity over the 22-nucleotide sequence of SEQ ID NO:8, and further includes a nucleotide sequence that is complementary to the 22-nucleotide core sequence. The complementary sequence will have a length of from about 18 nucleotides to about 26 nucleotides, and will have a nucleotide sequence that has from 80% to 85%, from 85% to 90%, from 90% to 95%, 96%, 97%, 98%, 99%, or 100%, nucleotide sequence identity to the 22-nucleotide core sequence. The 22-nucleotide core sequence and the complementary sequence are separated from one another by 1 nucleotide, 2 nucleotides (nt), 3 nt, 4 nt, 5 nt, 6 nt, 7 nt, 8 nt, 9 nt, 10 nt, 11 nt, 12 nt, 13 nt, 14 nt, 15 nt, 15-20 nt, 20-25 nt, or more than 25 nt.

A suitable miR-133-encoding nucleic acid comprises a nucleotide sequence encoding an miR-133 nucleic acid as described above. In some embodiments, an miR-133-encoding nucleic acid is contained within an expression vector. In some embodiments, a nucleotide sequence encoding an miR-133 nucleic acid is operably linked to a transcriptional regulatory element, e.g., a promoter, an enhancer, etc.

### Expression Vectors and Control Elements

As noted above, in some embodiments, a subject method involves introducing into a stem cell or a progenitor cell (or a population of stem cells or progenitor cells) a miR-1-encoding nucleic acid or an miR-133-encoding nucleic acid. In some embodiments, a subject method involves introducing into a stem cell or a progenitor cell (or a population of stem cells or progenitor cells) one or more nucleic acids comprising nucleotide sequences encoding miR-1 and miR-133. Suitable nucleic acids comprising miR-1-encoding and/or miR-133-encoding nucleotide sequences include expression vectors (“expression constructs”), where an expression vector comprising a miR-1-encoding and/or a miR-133-encoding nucleotide sequence is a “recombinant expression vector.”

In some embodiments, the expression construct is a viral construct, e.g., a recombinant adeno-associated virus construct (see, e.g., U.S. Pat. No. 7,078,387), a recombinant adenoviral construct, a recombinant lentiviral construct, etc.

Suitable expression vectors include, but are not limited to, viral vectors (e.g. viral vectors based on vaccinia virus; poliovirus; adenovirus (see, e.g., Li et al., Invest Opthalmol V is Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jornary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a miR-1-encoding nucleotide sequence is operably linked to a control element, e.g., a transcriptional control element, such as a promoter. Likewise, in some embodiments, a miR-133-encoding nucleotide sequence is operably linked to a control element, e.g., a transcriptional control element, such as a promoter. The transcriptional control element is functional in a eukaryotic cell, e.g., a mammalian cell.

Non-limiting examples of suitable eukaryotic promoters (promoters functional in a eukaryotic cell) include CMV immediate early, HSV thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

In some embodiments, the miR-1-encoding nucleotide sequence and/or the miR-133-encoding nucleotide sequence is operably linked to a cardiac-specific transcriptional regulator element (TRE), where TREs include promoters and enhancers. Suitable TREs include, but are not limited to, TREs derived from the following genes: myosin light chain-2, α-myosin heavy chain, AE3, cardiac troponin C, and cardiac actin. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

In some embodiments, the miR-1-encoding nucleotide sequence and/or the miR-133-encoding nucleotide sequence is operably linked to an inducible promoter. In some embodiments, the miR-1-encoding nucleotide sequence and/or the miR-133-encoding nucleotide sequence is operably linked to a constitutive promoter.

Methods of introducing a nucleic acid into a host cell are known in the art, and any known method can be used to introduce a nucleic acid (e.g., an expression construct) into a stem cell or progenitor cell. Suitable methods include, e.g., infection, lipofection, electroporation, calcium phosphate precipitation, DEAE-dextran mediated transfection, liposome-mediated transfection, and the like.

Introducing a nucleic acid may also include contacting a host cell with a compound, small molecule, activating RNA, or other agent in order to force expression of the endogenous nucleic acid.

### Genetically Modified Cells

The present disclosure provides genetically modified host cells, including isolated genetically modified host cells, where a subject genetically modified host cell comprises (has been genetically modified with): 1) an exogenous miR-1 nucleic acid; 2) an exogenous miR-133 nucleic acid; 3) both exogenous miR-1 nucleic acid and exogenous miR-133 nucleic acid; 4) an exogenous nucleic acid comprising a nucleotide sequence encoding a miR-1 nucleic acid; 5) an exogenous nucleic acid comprising a nucleotide sequence encoding a miR-133 nucleic acid; or 6) one or more exogenous nucleic acids comprising nucleotide sequences encoding both a miR-1 nucleic acid and a miR-133 nucleic acid. A subject genetically modified cell is generated by genetically modifying a host cell one or more exogenous nucleic acids (e.g., 1) an exogenous miR-1 nucleic acid; 2) an exogenous miR-133 nucleic acid; 3) both exogenous miR-1 nucleic acid and exogenous miR-133 nucleic acid; 4) an exogenous nucleic acid comprising a nucleotide sequence encoding a miR-1 nucleic acid; 5) an exogenous nucleic acid comprising a nucleotide sequence encoding a miR-133 nucleic acid; or 6) one or more exogenous nucleic acids comprising nucleotide sequences encoding both a miR-1 nucleic acid and a miR-133 nucleic acid). In some embodiments, a subject genetically modified host cell is in vitro. In some embodiments, a subject genetically modified host cell is a human cell or is derived from a human cell. In some embodiments, a subject genetically modified host cell is a rodent cell or is derived from a rodent cell. The present disclosure further provides progeny of a subject genetically modified stem cell or progenitor cell, where the progeny can comprise the same exogenous nucleic acid as the subject genetically modified stem cell or progenitor cell from which it was derived. The present disclosure further provides cardiomyocytes derived from a subject genetically modified stem cell or progenitor cell. The present disclosure further provides a composition comprising a subject genetically modified host cell.

### Genetically Modified Stem Cells and Genetically Modified Progenitor Cells

In some embodiments, a subject genetically modified host cell is a genetically modified stem cell or progenitor cell. Suitable host cells include, e.g., stem cells (adult stem cells, embryonic stem cells; iPS cells) and progenitor cells (including cardiac progenitor cells). Suitable host cells include mammalian stem cells and progenitor cells, including, e.g., rodent stem cells, rodent progenitor cells, human stem cells, human progenitor cells, etc. Suitable host cells include in vitro host cells, e.g., isolated host cells.

In some embodiments, a subject genetically modified host cell comprises an exogenous miR-1 nucleic acid. In some embodiments, a subject genetically modified host cell comprises an exogenous miR-133 nucleic acid. In some embodiments, a subject genetically modified host cell comprises both an exogenous miR-1 nucleic acid and an exogenous miR-133 nucleic acid. In some embodiments, a subject genetically modified host cell comprises an exogenous nucleic acid comprising a nucleotide sequence encoding a miR-1 nucleic acid, as described above. In other embodiments, a subject genetically modified host cell comprises an exogenous nucleic acid comprising a nucleotide sequence encoding a miR-1 nucleic acid, as described above. In other embodiments, a subject genetically modified host cell comprises one or more exogenous nucleic acids comprising nucleotide sequences encoding both a miR-1 nucleic acid and a miR-133 nucleic acid.

The present disclosure also provides a cardiomyocyte derived from a subject genetically modified stem cell or progenitor cell.

### Genetically Modified Cardiac Progenitor Cells; Genetically Modified Cardiomyocytes

The present disclosure provides a genetically modified cardiac progenitor cell comprising an exogenous miR-1 nucleic acid, or an exogenous nucleic acid comprising a nucleotide sequence encoding a miR-1 nucleic acid. The present disclosure provides a genetically modified cardiomyocyte comprising an exogenous miR-1 nucleic acid, or an exogenous nucleic acid comprising a nucleotide sequence encoding a miR-1 nucleic acid. The present disclosure provides a genetically modified cardiac progenitor cell comprising an exogenous miR-133 nucleic acid, or an exogenous nucleic acid comprising a nucleotide sequence encoding a miR-133 nucleic acid. The present disclosure provides a genetically modified cardiomyocyte comprising an exogenous miR-133 nucleic acid, or an exogenous nucleic acid comprising a nucleotide sequence encoding a miR-133 nucleic acid.

In some embodiments, the disclosure provides human or murine cells (e.g., cardiac progenitor cells or cardiomyocytes) comprising an exogenous miR-1 nucleic acid, or an exogenous nucleic acid comprising a nucleotide sequence encoding a miR-1 nucleic acid. In another aspect, the disclosure provides human or murine cells (e.g., cardiac progenitor cells or cardiomyocytes) comprising an exogenous miR-133 nucleic acid, or an exogenous nucleic acid comprising a nucleotide sequence encoding a miR-133 nucleic acid.

In some embodiments, the disclosure provides human or murine cells (e.g., cardiac progenitor cells or cardiomyocytes) derived from iPS cells. In some aspects, the human or murine cells (e.g., cardiac progenitor cells or cardiomyocytes) are generated following the introduction of a miR-1 nucleic acid, or an miR-1-encoding nucleic acid, into an iPS cell. In other aspects, the human or murine cells (e.g., cardiac progenitor cells or cardiomyocytes) are generated following the introduction of a miR-133 nucleic acid, or an miR-133-encoding nucleic acid, into an iPS cell.

### Exogenous Nucleic Acids

As noted above, a subject genetically modified host cell comprises an exogenous nucleic acid. For simplicity, “exogenous nucleic acid” is used to refer to: 1) an exogenous miR-1 nucleic acid; 2) an exogenous miR-133 nucleic acid; 3) both exogenous miR-1 nucleic acid and exogenous miR-133 nucleic acid; 4) an exogenous nucleic acid comprising a nucleotide sequence encoding a miR-1 nucleic acid; 5) an exogenous nucleic acid comprising a nucleotide sequence encoding a miR-133 nucleic acid; or 6) one or more exogenous nucleic acids comprising nucleotide sequences encoding both a miR-1 nucleic acid and a miR-133 nucleic acid.

In any of the above-described embodiments, the exogenous nucleic acid (e.g., 1) an exogenous miR-1 nucleic acid; 2) an exogenous miR-133 nucleic acid; 3) both exogenous miR-1 nucleic acid and exogenous miR-133 nucleic acid; 4) an exogenous nucleic acid comprising a nucleotide sequence encoding a miR-1 nucleic acid; 5) an exogenous nucleic acid comprising a nucleotide sequence encoding a miR-133 nucleic acid; or 6) one or more exogenous nucleic acids comprising nucleotide sequences encoding both a miR-1 nucleic acid and a miR-133 nucleic acid) is stably integrated into the genome of the host cell. In any of the above-described embodiments, the exogenous nucleic acid (e.g., 1) an exogenous miR-1 nucleic acid; 2) an exogenous miR-133 nucleic acid; 3) both exogenous miR-1 nucleic acid and exogenous miR-133 nucleic acid; 4) an exogenous nucleic acid comprising a nucleotide sequence encoding a miR-1 nucleic acid; 5) an exogenous nucleic acid comprising a nucleotide sequence encoding a miR-133 nucleic acid; or 6) one or more exogenous nucleic acids comprising nucleotide sequences encoding both a miR-1 nucleic acid and a miR-133 nucleic acid) is not integrated into the genome of the host cell and is instead present extrachromosomally.

In some embodiments, the exogenous nucleic acid is a recombinant expression vector. In some embodiments, the exogenous nucleic acid is a recombinant expression vector and is stably integrated into the genome of the host cell. For example, in some embodiments, an exogenous miR-1 nucleic acid, or an exogenous nucleic acid comprising a nucleotide sequence encoding a miR-1 nucleic acid, is present in a lentivirus vector, and the recombinant lentivirus vector is stably integrated into the genome of the host cell (e.g., stem cell; progenitor cell; cardiac progenitor cell; cardiomyocyte).

Methods of introducing a nucleic acid into a host cell are known in the art, and any known method can be used to introduce a nucleic acid (e.g., an expression construct) into a host cell. Suitable methods include, e.g., infection, lipofection, electroporation, calcium phosphate precipitation, DEAE-dextran mediated transfection, liposome-mediated transfection, and the like.

### Compositions

The present disclosure provides a composition comprising a subject genetically modified host cell. A subject composition comprises a subject genetically modified host cell; and will in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol, dimethylsulfoxide, etc.; nutritional media appropriate to the cell; and the like.

In some embodiments, a subject composition comprises a subject genetically modified host cell and a matrix (a “subject genetically modified cell/matrix composition”), where a subject genetically modified host cell is associated with the matrix. The term “matrix” refers to any suitable carrier material to which the genetically modified cells are able to attach themselves or adhere in order to form a cell composite. In some embodiments, the matrix or carrier material is present already in a three-dimensional form desired for later application. For example, bovine pericardial tissue is used as matrix which is crosslinked with collagen, decellularized and photofixed.

For example, a matrix (also referred to as a “biocompatible substrate”) is a material that is suitable for implantation into a subject. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate can provide the supportive framework that allows cells to attach to it and grow on it.

Suitable matrix components include, e.g., collagen; gelatin; fibrin; fibrinogen; laminin; a glycosaminoglycan; elastin; hyaluronic acid; a proteoglycan; a glycan; poly(lactic acid); poly(vinyl alcohol); poly(vinyl pyrrolidone); poly(ethylene oxide); cellulose; a cellulose derivative; starch; a starch derivative; poly(caprolactone); poly(hydroxy butyric acid); mucin; and the like. In some embodiments, the matrix comprises one or more of collagen, gelatin, fibrin, fibrinogen, laminin, and elastin; and can further comprise a non-proteinaceous polymer, e.g., can further comprise one or more of poly(lactic acid), poly(vinyl alcohol), poly(vinyl pyrrolidone), poly(ethylene oxide), poly(caprolactone), poly(hydroxy butyric acid), cellulose, a cellulose derivative, starch, and a starch derivative. In some embodiments, the matrix comprises one or more of collagen, gelatin, fibrin, fibrinogen, laminin, and elastin; and can further comprise hyaluronic acid, a proteoglycan, a glycosaminoglycan, or a glycan. Where the matrix comprises collagen, the collagen can comprise type I collagen, type II collagen, type III collagen, type V collagen, type XI collagen, and combinations thereof.

The matrix can be a hydrogel. A suitable hydrogel is a polymer of two or more monomers, e.g., a homopolymer or a heteropolymer comprising multiple monomers. Suitable hydrogel monomers include the following: lactic acid, glycolic acid, acrylic acid, 1-hydroxyethyl methacrylate (HEMA), ethyl methacrylate (EMA), propylene glycol methacrylate (PEMA), acrylamide (AAM), N-vinylpyrrolidone, methyl methacrylate (MMA), glycidyl methacrylate (GDMA), glycol methacrylate (GMA), ethylene glycol, fumaric acid, and the like. Common cross linking agents include tetraethylene glycol dimethacrylate (TEGDMA) and N,N′-methylenebisacrylamide. The hydrogel can be homopolymeric, or can comprise co-polymers of two or more of the aforementioned polymers. Exemplary hydrogels include, but are not limited to, a copolymer of poly(ethylene oxide) (PEO) and poly(propylene oxide) (PPO); Pluronic™ F-127 (a difunctional block copolymer of PEO and PPO of the nominal formula EO₁₀₀-PO₆₅-EO₁₀₀, where EO is ethylene oxide and PO is propylene oxide); poloxamer 407 (a tri-block copolymer consisting of a central block of poly(propylene glycol) flanked by two hydrophilic blocks of poly(ethylene glycol)); a poly(ethylene oxide)-poly(propylene oxide)-poly(ethylene oxide) co-polymer with a nominal molecular weight of 12,500 Daltons and a PEO:PPO ratio of 2:1); a poly(N-isopropylacrylamide)-base hydrogel (a PNIPAAm-based hydrogel); a PNIPAAm-acrylic acid co-polymer (PNIPAAm-co-AAc); poly(2-hydroxyethyl methacrylate); poly(vinyl pyrrolidone); and the like.

A subject genetically modified cell/matrix composition can further comprise one or more additional components, where suitable additional components include, e.g., a growth factor; an antioxidant; a nutritional transporter (e.g., transferrin); a polyamine (e.g., glutathione, spermidine, etc.); and the like.

The cell density in a subject genetically modified cell/matrix composition can range from about 10² cells/mm³ to about 10⁹ cells/mm³, e.g., from about 10² cells/mm³ to about 10⁴ cells/mm³, from about 10⁴ cells/mm³ to about 10⁶ cells/mm³, from about 10⁶ cells/mm³ to about 10⁷ cells/mm³, from about 10⁷ cells/mm³ to about 10⁸ cells/mm³, or from about 10⁸ cells/mm³ to about 10⁹ cells/mm³.

The matrix can take any of a variety of forms, or can be relatively amorphous. For example, the matrix can be in the form of a sheet, a cylinder, a sphere, etc.

Separating Cardiomyocytes or Cardiac Progenitors from a Mixed Cell Population

In some embodiments, a subject method comprises: a) inducing cardiomyogenesis in a population of stem cells or progenitor cells, generating a mixed population of undifferentiated stem cells and/or undifferentiated progenitor cells and cardiomyocytes; and b) separating cardiomyocytes from the undifferentiated (non-cardiomyocyte) cells. In some embodiments, the separation step comprises contacting the cells with an antibody specific for a cardiomyocyte-specific cell surface marker. Suitable cardiomyocyte-specific cell surface markers include, but are not limited to, troponin, tropomyosin, N-cadherin, and CD166.

Alternatively, non-cardiomyocytes can be removed from a mixed population comprising cardiomyocytes and non-cardiomyocytes, using one or more antibodies specific for cell-surface markers present on a non-cardiomyocyte cell.

In some embodiments, a subject method comprises: a) inducing cardiomyogenesis in a population of stem cells, generating a mixed population of undifferentiated stem cells and/or non-cardiac progenitor cells and cardiac progenitors; and b) separating cardiac progenitors from the undifferentiated (non-cardiomyocyte) cells or non-cardiac progenitors.

Separation can be carried out using well-known methods, including, e.g., any of a variety of sorting methods, e.g., fluorescence activated cell sorting (FACS), negative selection methods, etc. The selected cells are separated from non-selected cells, generating a population of selected (“sorted”) cells. A selected cell population can be at least about 75%, at least about 80%, at least about 85%, at least about 90%, at least about 95%, at least about 98%, at least about 99%, or greater than 99% cardiomyocytes.

Cell sorting (separation) methods are well known in the art. Procedures for separation may include magnetic separation, using antibody-coated magnetic beads, affinity chromatography and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. Dead cells may be eliminated by selection with dyes associated with dead cells (propidium iodide [PI]). Any technique may be employed which is not unduly detrimental to the viability of the selected cells. Where the selection involves use of one or more antibodies, the antibodies can be conjugated with labels to allow for ease of separation of the particular cell type, e.g. magnetic beads; biotin, which binds with high affinity to avidin or streptavidin; fluorochromes, which can be used with a fluorescence activated cell sorter; haptens; and the like. Multi-color analyses may be employed with the FACS or in a combination of immunomagnetic separation and flow cytometry.

### Utility

A subject method is useful for generating a population of cardiomyocytes or cardiac progenitors, which cardiomyocytes or cardiac progenitors can be used in analytical assays, for generating artificial heart tissue, and in treatment methods.

### Analytical Assays

A subject method can be used to generate cardiomyocytes or cardiac progenitors for analytical assays. Analytical assays include, e.g., introduction of the cardiomyocytes or cardiac progenitors into a non-human animal model of a disease (e.g., a cardiac disease) to determine efficacy of the cardiomyocytes or cardiac progenitors in the treatment of the disease; use of the cardiomyocytes in screening methods to identify candidate agents suitable for use in treating cardiac disorders; and the like. In some cases, a cardiomyocyte or cardiac progenitor generated using a subject method can be used to assess the toxicity of a test agent or for drug optimization. In some cases, cardiac progenitor cells generated using a subject method may be used to screen for agents that induce maturation of a cardiac progenitor cell to a more highly differentiated cell, e.g. a cardiomyocyte.

### Animal Models

In some embodiments, a cardiomyocyte or cardiac progenitor generated using a subject method can be introduced into a non-human animal model of a cardiac disorder, and the effect of the cardiomyocyte or cardiac progenitor on ameliorating the disorder can be tested in the non-human animal model (e.g., a rodent model such as a rat model, a guinea pig model, a mouse model, etc.; a non-human primate model; a lagomorph model; and the like). For example, the effect of a cardiomyocyte or cardiac progenitor generated using a subject method on a cardiac disorder in a non-human animal model of the disorder can be tested by introducing the cardiomyocyte or cardiac progenitor into, near, or around diseased cardiac tissue in the non-human animal model; and the effect, if any, of the introduced cardiomyocyte or cardiac progenitor on cardiac function can be assessed. Methods of assessing cardiac function are well known in the art; and any such method can be used.

### Drug/Agent Screening or Identification

Cardiac progenitor cells or cardiomyocytes generated using a subject method may be used to screen for drugs or test agents (e.g., solvents, small molecule drugs, peptides, oligonucleotides) or environmental conditions (e.g., culture conditions or manipulation) that affect the characteristics of such cells and/or their various progeny. See, e.g., U.S. Pat. No. 7,425,448, incorporated herein by reference in its entirety. Drugs or test agents may be individual small molecules of choice (e.g., a lead compound from a previous drug screen) or in some cases, the drugs or test agents to be screened come from a combinatorial library, e.g., a collection of diverse chemical compounds generated by either chemical synthesis or biological synthesis by combining a number of chemical “building blocks.” For example, a linear combinatorial chemical library such as a polypeptide library is formed by combining a set of amino acids in every possible way for a given compound length (e.g., the number of amino acids in a polypeptide compound). Millions of test agents (e.g., chemical compounds) can be synthesized through such combinatorial mixing of chemical building blocks. Indeed, theoretically, the systematic, combinatorial mixing of 100 interchangeable chemical building blocks results in the synthesis of 100 million tetrameric compounds or 10 billion pentameric compounds. See, e.g., Gallop et al. (1994), J. Med. Chem. 37(9), 1233. Preparation and screening of combinatorial chemical libraries are well known in the art. Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell et al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514).

Numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

In some embodiments, a cardiomyocyte or cardiac progenitor generated using a subject method is contacted with a test agent, and the effect, if any, of the test agent on a biological activity of the cardiomyocyte or cardiac progenitor is assessed, where a test agent that has an effect on a biological activity of the cardiomyocyte or cardiac progenitor is a candidate agent for treating a cardiac disorder or condition. For example, a test agent of interest is one that increases a biological activity of the cardiomyocyte or cardiac progenitor by at least about 5%, at least about 10%, at least about 15%, at least about 20%, at least about 25%, at least about 30%, at least about 40%, at least about 50%, at least about 75%, at least about 2-fold, at least about 2.5-fold, at least about 5-fold, at least about 10-fold, or more than 10-fold, compared to the biological activity in the absence of the test agent. A test agent of interest is a candidate agent for treating a cardiac disorder or condition. In some embodiments, the contacting is carried out in vitro. In other embodiments, the contacting is carried out in vivo, e.g, in an non-human animal.

A “biological activity” includes, e.g., one or more of marker expression (e.g., cardiomyocyte-specific marker expression), receptor binding, ion channel activity, contractile activity, and electrophysiological activity.

For example, in some embodiments, the effect, if any, of the test agent on expression of a cardiomyocyte marker is assessed. Cardiomyocyte markers include, e.g., cardiac troponin I (cTnI), cardiac troponin T (cTnT), sarcomeric myosin heavy chain (MHC), GATA-4, Nkx2.5, N-cadherin, β-adrenoceptor (β1-AR), a member of the MEF-2 family of transcription factors, creatine kinase MB (CK-MB), myoglobin, and atrial natriuretic factor (ANF).

As another example, the effect, if any, of the test agent on electrophysiology of the cardiomyocyte or cardiac progenitor is assessed. Electrophysiology can be studied by patch clamp analysis for cardiomyocyte-like action potentials. See Igelmund et al., Pflugers Arch. 437:669, 1999; Wobus et al., Ann. N.Y. Acad. Sci. 27:752, 1995; and Doevendans et al., J. Mol. Cell. Cardiol. 32:839, 2000.

As another example, in some embodiments, the effect, if any, of the test agent on ligand-gated ion channel activity is assessed. As another example, in some embodiments, the effect, if any, of the test agent on voltage-gated ion channel activity is assessed. The effect of a test agent on ion channel activity is readily assessed using standard assays, e.g., by measuring the level of an intracellular ion (e.g., Na⁺, Ca²⁺, K⁺, etc.). A change in the intracellular concentration of an ion can be detected using an indicator appropriate to the ion whose influx is controlled by the channel. For example, where the ion channel is a potassium ion channel, a potassium-detecting dye is used; where the ion channel is a calcium ion channel, a calcium-detecting dye is used; etc.

Suitable intracellular K⁺ ion-detecting dyes include, but are not limited to, K⁺-binding benzofuran isophthalate and the like.

Suitable intracellular Ca²⁺ ion-detecting dyes include, but are not limited to, fura-2, bis-fura 2, indo-1, Quin-2, Quin-2 AM, Benzothiaza-1, Benzothiaza-2, indo-5F, Fura-FF, BTC, Mag-Fura-2, Mag-Fura-5, Mag-Indo-1, fluo-3, rhod-2, fura-4F, fura-5F, fura-6F, fluo-4, fluo-5F, fluo-5N, Oregon Green 488 BAPTA, Calcium Green, Calcein, Fura-C18, Calcium Green-C18, Calcium Orange, Calcium Crimson, Calcium Green-5N, Magnesium Green, Oregon Green 488 BAPTA-1, Oregon Green 488 BAPTA-2, X-rhod-1, Fura Red, Rhod-5F, Rhod-5N, X-Rhod-5N, Mag-Rhod-2, Mag-X-Rhod-1, Fluo-5N, Fluo-5F, Fluo-4FF, Mag-Fluo-4, Aequorin, dextran conjugates or any other derivatives of any of these dyes, and others (see, e.g., the catalog or Internet site for Molecular Probes, Eugene, see, also, Nuccitelli, ed., Methods in Cell Biology, Volume 40: A Practical Guide to the Study of Calcium in Living Cells, Academic Press (1994); Lambert, ed., Calcium Signaling Protocols (Methods in Molecular Biology Volume 114), Humana Press (1999); W. T. Mason, ed., Fluorescent and Luminescent Probes for Biological Activity. A Practical Guide to Technology for Quantitative Real-Time Analysis, Second Ed, Academic Press (1999); Calcium Signaling Protocols (Methods in Molecular Biology), 2005, D. G. Lamber, ed., Humana Press.)

In some embodiments, screening of test agents is conducted in cardiomyocytes or cardiac progenitors generated using a subject method and displaying an abnormal cellular phenotype (e.g., abnormal cell morphology, gene expression, or signaling), associated with a health condition or a predisposition to the health condition. Such assays may include contacting a test population of cardiomyocytes or cardiac progenitors generated using a subject method (e.g., generated from one or more iPS donors exhibiting a cardiac condition described herein) with a test compound and contacting with a negative control compound a negative control population of cardiomyocytes or cardiac progenitors generated using a subject method (e.g., generated from one or more iPS donors exhibiting a cardiac or cardiovascular condition described herein, e.g., coronary artery disease, cardiac myopathy, aneurysm, angina, atherosclerosis, etc.). The assayed cellular phenotype associated with the health condition of interest in the test and negative control populations can then be compared to a normal cellular phenotype. Where the assayed cellular phenotype in the test population is determined as being closer to a normal cellular phenotype than that exhibited by the negative control population, the drug candidate compound is identified as normalizing the phenotype.

The effect of a test agent in the assays described herein can be assessed using any standard assay to observe phenotype or activity of cardiomyocytes or cardiac progenitors generated using a subject method, such as marker expression, receptor binding, contractile activity, or electrophysiology—either in cell culture or in vivo. See, e.g., U.S. Pat. No. 7,425,448. For example, pharmaceutical candidates are tested for their effect on contractile activity—such as whether they increase or decrease the extent or frequency of contraction, using any methods known in the art. Where an effect is observed, the concentration of the compound can be titrated to determine the median effective dose (ED50).

### Test Agent/Drug Toxicity

The cardiomyocyte and/or cardiac progenitor generated using a subject method can be used to assess the toxicity of a test agent, or drug, e.g., a test agent or drug designed to have a pharmacological effect on cardiac progenitors or cardiomyocytes, e.g., a test agent or drug designed to have effects on cells other than cardiac progenitors or cardiomyocytes but potentially affecting cardiac progenitors or cardiomyocytes as an unintended consequence. In some embodiments, the disclosure provides methods for evaluating the toxic effects of a drug, test agent, or other factor, in a human or non-human (e.g., murine; lagomorph; non-human primate) subject, comprising contacting one or more cardiomyocytes or cardiac progenitors generated using a subject method with a dose of a drug, test agent, or other factor and assaying the contacted cardiac progenitor cells and/or cardiomyocytes for markers of toxicity or cardiotoxicity.

Any method known in the art may be used to evaluate the toxicity or adverse effects of a test agent or drug on cardiomyocytes or cardiac progenitors generated using a subject method. Cytotoxicity or cardiotoxicity can be determined, e.g., by the effect on cell viability, survival, morphology, and the expression of certain markers and receptors. For example, biochemical markers of myocardial cell necrosis (e.g., cardiac troponin T and I (cTnT, cTnI)) may be used to assess drug-induced toxicity or adverse reactions in cardiomyocytes or cardiac progenitors generated using a subject method, where the presence of such markers in extracellular fluid (e.g., cell culture medium) can indicate necrosis. See, e.g., Gaze and Collinson (2005) Expert Opin Drug Metab Toxicol 1(4):715-725. In another example, lactate dehydrogenase is used to assess drug-induced toxicity or adverse reactions in cardiomyocytes or cardiac progenitors generated using a subject method. See, e.g., Inoue et al. (2007) AATEX 14, Special Issue: 457-462. In another example, the effects of a drug on chromosomal DNA can be determined by measuring DNA synthesis or repair and used to assess drug-induced toxicity or adverse reactions in cardiomyocytes or cardiac progenitors generated using a subject method. In still another example, the rate, degree, and/or timing of [³H]-thymidine or BrdU incorporation may be evaluated to assess drug-induced toxicity or adverse reactions in cardiomyocytes or cardiac progenitors generated using a subject method. In yet another example, evaluating the rate or nature of sister chromatid exchange, determined by metaphase spread, can be used to assess drug-induced toxicity or adverse reactions in cardiomyocytes or cardiac progenitors generated using a subject method. See, e.g., A. Vickers (pp 375-410 in In vitro Methods in Pharmaceutical Research, Academic Press, 1997). In yet another example, assays to measure electrophysiology or activity of ion-gated channels (e.g., Calcium-gated channels) can be used to assess drug-induced toxicity or adverse reactions in cardiomyocytes or cardiac progenitors generated using a subject method. In still another example, contractile activity (e.g., frequency of contraction) can be used to assess drug-induced toxicity or adverse reactions in cardiomyocytes or cardiac progenitors generated using a subject method.

In some embodiments, the present disclosure provides methods for reducing the risk of drug toxicity in a human or murine subject, comprising contacting one or more cardiomyocytes or cardiac progenitors generated using a subject method with a dose of a drug, test agent, or pharmacological agent, assaying the contacted one or more differentiated cells for toxicity, and prescribing or administering the pharmacological agent to the subject if the assay is negative for toxicity in the contacted cells. In some embodiments, the present disclosure provides methods for reducing the risk of drug toxicity in a human or murine subject, comprising contacting one or more cardiomyocytes or cardiac progenitors generated using a subject method with a dose of a pharmacological agent, assaying the contacted one or more differentiated cells for toxicity, and prescribing or administering the pharmacological agent to the subject if the assay indicates a low risk or no risk for toxicity in the contacted cells.

### Screen for Maturation Agents

In some applications, cardiac progenitors generated using a subject method are used to screen drugs, test agents or other factors that promote maturation into later-stage cardiomyocyte precursors, or terminally differentiated cells (e.g., cardiomyocytes), or to promote proliferation and maintenance of such cells in long-term culture. For example, candidate maturation drugs, test agents, factors or growth factors are tested by adding them to cells in different wells, and then determining any phenotypic change that results, according to desirable criteria for further culture and use of the cells.

### Treatment Methods

A subject method is useful for generating artificial heart tissue, e.g., for implanting into a mammalian subject in need thereof. A subject method is useful for replacing damaged heart tissue (e.g., ischemic heart tissue). A subject method is useful for stimulating endogenous stem cells resident in the heart to undergo cardiomyogenesis. Where a subject method involves introducing (implanting) a cardiomyocyte into an individual, allogenic or autologous transplantation can be carried out.

The present disclosure provides methods of treating a cardiac disorder in an individual, the method generally involving administering to an individual in need thereof a therapeutically effective amount of: a) a population of cardiomyocytes prepared using a subject method; b) a population of cardiac progenitors prepared using a subject method; or c) an artificial heart tissue prepared using a subject method.

For example, in some embodiments, a subject method comprises: i) inducing a stem cell to differentiate into a cardiomyocyte; and ii) introducing the cardiomyocyte into an individual in need thereof. In other embodiments, a subject method comprises: i) inducing a stem cell to differentiate into a cardiac progenitor (e.g., using miR-133); ii) inducing the cardiac progenitor to differentiate into a cardiomyocyte (e.g., using miR-1); and iii) introducing the cardiomyocyte into an individual in need thereof.

In other embodiments, a subject method comprises: i) generating artificial heart tissue by: a) inducing a stem cell to differentiate into a cardiomyocyte; and b) associating the cardiomyocyte with a matrix, to form artificial heart tissue; and ii) introducing the artificial heart tissue into an individual in need thereof. In other embodiments, a subject comprises: i) generating artificial heart tissue by: a) inducing a stem cell to differentiate into a cardiomyocyte, where the stem cell is associated with a matrix, and the cardiomyocyte is also associated with a matrix, thereby generating artificial heart tissue comprising the matrix-associated cardiomyocyte; and ii) introducing the artificial heart tissue into an individual in need thereof. The artificial heart tissue can be introduced into, on, or around existing heart tissue in the individual.

In other embodiments, a subject method comprises: i) generating an iPS cell from a somatic cell from an individual; ii) inducing the iPS cell to differentiate into a cardiomyocyte; and iii) introducing the cardiomyocyte into the individual from whom the somatic cell was obtained, which individual is in need of a cardiomyocyte. In other embodiments, a subject method comprises: i) generating an iPS cell from a somatic cell from a donor individual; ii) inducing the iPS cell to differentiate into a cardiomyocyte; and iii) introducing the cardiomyocyte into a recipient individual, where the recipient individual not the same individual as the donor individual, which recipient individual is in need of a cardiomyocyte.

In some embodiments, a subject method comprises: i) generating an iPS cell from a somatic cell from an individual; ii) inducing the iPS cell to differentiate into a cardiomyocyte; iii) associating the cardiomyocyte with a matrix, to generate artificial heart tissue; and iv) introducing the artificial heart tissue into the individual from whom the somatic cell was obtained, which individual is in need of the artificial heart tissue. In some embodiments, a subject method comprises: i) generating an iPS cell from a somatic cell from a donor individual; ii) inducing the iPS cell to differentiate into a cardiomyocyte; iii) associating the cardiomyocyte with a matrix, to generate artificial heart tissue; and iv) introducing the artificial heart tissue into a recipient individual (where the recipient individual is not the same individual as the donor individual), which recipient individual is in need of the artificial heart tissue.

In some embodiments, a subject method comprises: i) generating an iPS cell from a somatic cell from an individual (including but not limited to: a healthy individual, an individual suffering from a cardiac condition as described, e.g., herein; an individual with a congenital heart defect, as described, e.g., herein; an individual with coronary artery disease; an individual suffering from a degenerative muscle disease or condition; etc.); ii) inducing the iPS cell to differentiate into a cardiomyocyte, where the iPS cell is associated with a matrix, and the cardiomyocyte is also associated with a matrix, thereby generating artificial heart tissue comprising the matrix-associated cardiomyocyte; and iii) introducing the artificial heart tissue into the individual from whom the somatic cell was obtained, which individual is in need of the artificial heart tissue. In some embodiments, a subject method comprises: i) generating an iPS cell from a somatic cell from a donor individual (including but not limited to: a healthy individual, an individual suffering from a cardiac condition as described, e.g., herein, an individual with a congenital heart defect, as described, e.g., herein, an individual with coronary artery disease, or an individual suffering from a degenerative muscle disease or condition); ii) inducing the iPS cell to differentiate into a cardiomyocyte, where the iPS cell is associated with a matrix, and the cardiomyocyte is also associated with a matrix, thereby generating artificial heart tissue comprising the matrix-associated cardiomyocyte; and iii) introducing the artificial heart tissue into a recipient individual (where the recipient individual is not the same individual as the donor individual, where the recipient individual is a relative of the donor individual, or where the recipient individual is HLA-matched to the donor individual), which recipient individual is in need of the artificial heart tissue.

Individuals in need of treatment using a subject method and/or donor individuals include, but are not limited to, individuals having a congenital heart defect; individuals suffering from a degenerative muscle disease; individuals suffering from a condition that results in ischemic heart tissue, e.g., individuals with coronary artery disease; and the like. In some examples, a subject method is useful to treat a degenerative muscle disease or condition, e.g., familial cardiomyopathy, dilated cardiomyopathy, hypertrophic cardiomyopathy, restrictive cardiomyopathy, or coronary artery disease with resultant ischemic cardiomyopathy. In some examples, a subject method is useful to treat individuals having a cardiac or cardiovascular disease or disorder, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism.

Individuals who are suitable for treatment with a subject method and/or donor individuals include individuals (e.g., mammalian subjects, such as humans; non-human primates; experimental non-human mammalian subjects such as mice, rats, etc.) having a cardiac condition including but limited to a condition that results in ischemic heart tissue, e.g., individuals with coronary artery disease; and the like. In some examples, an individual suitable for treatment and/or a donor individual suffers from a cardiac or cardiovascular disease or condition, e.g., cardiovascular disease, aneurysm, angina, arrhythmia, atherosclerosis, cerebrovascular accident (stroke), cerebrovascular disease, congenital heart disease, congestive heart failure, myocarditis, valve disease coronary, artery disease dilated, diastolic dysfunction, endocarditis, high blood pressure (hypertension), cardiomyopathy, hypertrophic cardiomyopathy, restrictive cardiomyopathy, coronary artery disease with resultant ischemic cardiomyopathy, mitral valve prolapse, myocardial infarction (heart attack), or venous thromboembolism. In some examples, individuals suitable for treatment with a subject method and/or donor individuals include individuals who have a degenerative muscle disease, e.g., familial cardiomyopathy, dilated cardiomyopathy, hypertrophic cardiomyopathy, restrictive cardiomyopathy, or coronary artery disease with resultant ischemic cardiomyopathy.

For administration to a mammalian host, a cardiomyocyte population or cardiac progenitor cell population generated using a subject method can be formulated as a pharmaceutical composition. A pharmaceutical composition can be a sterile aqueous or non-aqueous solution, suspension or emulsion, which additionally comprises a physiologically acceptable carrier (i.e., a non-toxic material that does not interfere with the activity of the cardiomyocytes). Any suitable carrier known to those of ordinary skill in the art may be employed in a subject pharmaceutical composition. The selection of a carrier will depend, in part, on the nature of the substance (i.e., cells or chemical compounds) being administered. Representative carriers include physiological saline solutions, gelatin, water, alcohols, natural or synthetic oils, saccharide solutions, glycols, injectable organic esters such as ethyl oleate or a combination of such materials. Optionally, a pharmaceutical composition may additionally contain preservatives and/or other additives such as, for example, antimicrobial agents, anti-oxidants, chelating agents and/or inert gases, and/or other active ingredients.

In some embodiments, a cardiomyocyte population or cardiac progenitor population is encapsulated, according to known encapsulation technologies, including microencapsulation (see, e.g., U.S. Pat. Nos. 4,352,883; 4,353,888; and 5,084,350). Where the cardiomyocytes or cardiac progenitors are encapsulated, in some embodiments the cardiomyocytes or cardiac progenitors are encapsulated by macroencapsulation, as described in U.S. Pat. Nos. 5,284,761; 5,158,881; 4,976,859; 4,968,733; 5,800,828 and published PCT patent application WO 95/05452.

In some embodiments, a cardiomyocyte population or cardiac progenitor population is present in a matrix, as described below.

A unit dosage form of a cardiomyocyte population or cardiac progenitor population can contain from about 10³ cells to about 10⁹ cells, e.g., from about 10³ cells to about 10⁴ cells, from about 10⁴ cells to about 10⁵ cells, from about 10⁵ cells to about 10⁶ cells, from about 10⁶ cells to about 10⁷ cells, from about 10⁷ cells to about 10⁸ cells, or from about 10⁸ cells to about 10⁹ cells.

A cardiomyocyte population can be cryopreserved according to routine procedures. For example, cryopreservation can be carried out on from about one to ten million cells in “freeze” medium which can include a suitable proliferation medium, 10% BSA and 7.5% dimethylsulfoxide. Cells are centrifuged. Growth medium is aspirated and replaced with freeze medium. Cells are resuspended as spheres. Cells are slowly frozen, by, e.g., placing in a container at −80° C. Cells are thawed by swirling in a 37° C. bath, resuspended in fresh proliferation medium, and grown as described above.

### Artificial Heart Tissue

In some embodiments, a subject method comprises: a) inducing cardiomyogenesis in a population of stem cells or progenitor cells in vitro, e.g., where the stem cells or progenitor cells are present in a matrix, wherein a population of cardiomyocytes is generated; and b) implanting the population of cardiomyocytes into or on an existing heart tissue in an individual. Thus, the present disclosure provides a method for generating artificial heart tissue in vitro; and implanting the artificial heart tissue in vivo. In some embodiments, a subject method comprises: a) inducing cardiomyogenesis in a population of stem cells or progenitor cells in vitro, generating a population of cardiomyocytes; b) associating the cardiomyocytes with a matrix, forming an artificial heart tissue; and c) implanting the artificial heart tissue into or on an existing heart tissue in an individual.

The artificial heart tissue can be used for allogenic or autologous transplantation into an individual in need thereof. To produce artificial heart tissue, a matrix can be provided which is brought into contact with the stem cells or progenitor cells, where the stem cells or progenitor cells are induced to undergo cardiomyogenesis using a subject method, as described above. This means that this matrix is transferred into a suitable vessel and a layer of the cell-containing culture medium is placed on top (before or during the differentiation of the expanded stem cells or progenitor cells). The term “matrix” should be understood in this connection to mean any suitable carrier material to which the cells are able to attach themselves or adhere in order to form the corresponding cell composite, i.e. the artificial tissue. In some embodiments, the matrix or carrier material, respectively, is present already in a three-dimensional form desired for later application. For example, bovine pericardial tissue is used as matrix which is crosslinked with collagen, decellularized and photofixed.

For example, a matrix (also referred to as a “biocompatible substrate”) is a material that is suitable for implantation into a subject onto which a cell population can be deposited. A biocompatible substrate does not cause toxic or injurious effects once implanted in the subject. In one embodiment, the biocompatible substrate is a polymer with a surface that can be shaped into the desired structure that requires repairing or replacing. The polymer can also be shaped into a part of a structure that requires repairing or replacing. The biocompatible substrate provides the supportive framework that allows cells to attach to it, and grow on it. Cultured populations of cells can then be grown on the biocompatible substrate, which provides the appropriate interstitial distances required for cell-cell interaction.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Materials and Methods

### Mouse ES Cell Culture and Flow Cytometry

The mouse E14 embryonic stem (ES) cell line was maintained as a monolayer in medium supplemented with 10% fetal bovine serum, leukemia inhibitory factor (LIF)-conditioned medium, pyruvate, glutamine, and β-mercaptoethanol in gelatin-coated tissue-culture plates and passaged with trypsin. Cells were differentiated by the hanging drop method. Briefly, cells were trypsinized and resuspended at 25,000 cells/ml in differentiation medium (20% fetal bovine serum, pyruvate, glutamine, and β-mercaptoethanol). Droplets (20 μl) were transferred to each well of a 96-well v-bottom tissue culture plate, which was then inverted. After 2 days of incubation at 37° C., the plates were turned upright, and 200 μl of differentiation medium was added to each well. For neuroectodermal or endodermal induction, 0.5 μM retinoic acid (Sigma) or 50 ng/ml recombinant nodal (R&D Systems), respectively, was added to the wells 96 h after formation of the hanging drops. The medium was changed every 2 days. The β-myosin heavy chain (β-MHC)-green fluorescent protein (GFP) E14 cells were a gift of W. Tingley and R. Shaw. For flow cytometry studies, embryoid bodies (EBs) were dissociated via trypsin and passed through a nylon cell strainer. Flk-1⁺ cells were labeled with a phycoerythrin (PE)-conjugated Flk-1 antibody (BD Pharmingen) and a Becton Dickinson (Franklin Lakes, N.J.) fluorescence activated cell sorting (FACS) Diva flow cytometer and cell sorter was used for detecting and sorting Flk-1⁺, NRx2.5-GFP⁺, or βMHC-GFP⁺ cells.

miRNA and mRNA Expression Microarray Analyses

ES cells or EBs were harvested in Trizol (Invitrogen) for total RNA isolation. For mRNA expression microarray analysis, 1 μg total RNA was labeled and hybridized to a mouse mRNA expression microarray (Affymetrix). Gene expression values were obtained from Affymetrix CEL files using the GC-RMA package from Bioconductor (Dudoit et al. 2003; Wu et al. 2004). To identify transcripts differing in mean expression across the three experimental groups (mES^{wt}, mES^{miR-1}, and mES^{miR-133} EBs), p values were calculated by permutation test with the F-statistic function from the multtest package of Bioconductor (Dudoit et al. 2003) and at test comparing each miRNA-expressing group to wild-type EBs. Fold changes in transcript levels were calculated from the mean log2 expression values versus the mean of control EBs.

For miRNA expression microarray, 100 ng of total RNA from each sample was labeled with Cy3 or Cy5 using miRCURY™ LNA microRNA Power labeling kit (Exiqon) and then hybridized to miRCURY™ LNA arrays (Exiqon). Hybridization quality was assessed with Bioconductor marray package and log2 ratios of Cy5 to Cy3 signals were calculated with limma package.

### Quantitative RT-PCR

ES cells or EBs were harvested in Trizol (Invitrogen) for total RNA isolation. For mRNA quantitative reverse transcription-polymerase chain reaction (qRT-PCR), 2 μg of total RNA from each sample was reversed transcribed with Superscript III (Invitrogen). 1/16 of the reverse transcription reaction was used for subsequent PCRs, which were performed in duplicate on an ABI 7900HT instrument (Applied Biosystems) using Taqman primer probe sets (Applied Biosystems) for each gene of interest and a GAPDH endogenous control primer probe set for normalization. Each qRT-PCR was performed on at least 3 different experimental samples; representative results are shown as fold expression relative to undifferentiated ES cells. Error bars reflect a 95% confidence interval.

miRNA qRT-PCR was performed with miRNA Taqman Expression Assays (Applied Biosystems) and the miRNA Reverse Transcription kit (Applied Biosystems). For each miRNA analyzed, 10 ng of total RNA was reverse transcribed with a miRNA-specific primer. A ubiquitous miRNA, miR-16, was used as the endogenous control. Each qRT-PCR was performed on at least three different experimental samples; representative results are shown as fold expression relative to undifferentiated ES cells. Error bars indicate 95% confidence intervals.

### Lentiviral Production and ES Cell Infection

Lentiviruses for miRNA expression were generated with the ViraPower Promoterless Lentiviral Gateway Expression System with MultiSite Gateway Technology (Invitrogen). The EF-1α promoter was recombined into the pLenti vector upstream of a cassette containing either miR-1 or miR-133 pre-miRNA sequence with an additional ˜100 nucleotides flanking each end, which was cloned by PCR from a bacterial artificial chromosome containing the mouse genomic miR-1-2 or miR-133a-1 sequences. Details of virus production and introduction into ES cells can be found in Supplemental Methods.

### Teratoma Formation

Teratomas were formed by subcutaneous injection of approximately 1×10⁶ control or miRNA-expressing mES cells into the rear flank of 8-week-old male SCID mice (n=10 mice per cell line). Transplanted cells of each line formed teratomas in the recipients and were analyzed 6 weeks after inoculation.

### Immunostaining

For immunocytochemistry studies, ES cells were plated on gelatinized cover slips and allowed to settle, rinsed with phosphate buffered saline (PBS), fixed in 4% paraformaldehyde for 1 h at room temperature with shaking, and stored in PBS at 4° C. The fixed cells were rinsed in PBS, blocked in blocking solution (1% bovine serum albumin, 1% Tween-20, and PBS) for 30 min at room temperature and incubated in primary antibody in a humidified chamber for 1 h at room temperature. The antibodies were diluted in blocking buffer as follows: Dll-1, 1:100 (AbCam, ab10554); Jag-1, 1:100 (AbCam, ab7771); Dll-4, 1:50 (AbCam, ab7280). After washing in PBS, the cells were incubated for 1 h with fluorescein isothiocyanate (FITC)-conjugated secondary:antibodies (1:200) at room temperature in a darkened chamber, rinsed with PBS, and mounted on slides with Vectashield containing 4′,6-diamidino-2-phenylindole (DAPI) (Vector Laboratories).

For immunohistochemical studies, teratomas were submerged in CPT (Sakuro), flash frozen in liquid nitrogen, and sectioned. Details of immunostaining and antibodies are in Supplemental Methods.

For EB immunohistochemistry, EBs were fixed in 4% paraformaldehyde, blocked in 5% goat serum, and incubated overnight in βIII-tubulin antibody (1:100; Chemicon, CBL412). The following day, EBs were rinsed, placed in rhodamine-conjugated anti-mouse IgG diluted 1:400 for 2 h, rinsed, mounted with Vectashield containing DAPI (Vector Laboratories), and visualized.

### Dll-1 Knockdown

mES cells were infected with lentiviral constructs encoding short hairpin RNAs (shRNAs) against mouse Dll-1 or a control shRNA (Sigma). After transduction and 2 days of recovery, infected mES cells were selected for 7 days with 1 μg/ml puromycin. Colonies were isolated, expanded, and assayed for Dll-1 knockdown compared to control-infected mES cells by qRT-PCR. The pluripotency of the resulting cell lines was assessed by measuring the proliferation rate and Oct3/4 expression and comparing the value to those of uninfected mES cells. Only lines that maintained normal levels of Oct3/4 expression and normal proliferation rates were used for further study.

miR-1 Target Analyses

12-well plates of Cos-1 cells were transfected for either luciferase assays or transient expression analyses using Lipofectamine 2000 (Invitrogen). For luciferase assays, a luciferase expression construct containing the 3′UTR of mouse Dll-1 (50 ng) was co-transfected alone or with miR-1 or miR-133 expression constructs (300 ng) and a LacZ expression construct. Empty expression plasmid was used to normalize the total DNA mass. After 24 hours, cells were harvested and the luciferase assays were performed using a Luciferase Assay Kit (Promega). β-galactosidase assays were also performed and the results were used to normalize for transfection efficiency. For transient expression analyses, a Dll-1 expression construct lacking Dll-1-derived 5′UTR sequence elements, but with the full mouse Dll-1 3′UTR and an n-terminal V5 epitope tag (75 ng) was co-transfected with increasing amounts of miR-1 expression construct (0 ng, 350 ng, or 700 ng). Empty expression vector was included to ensure equal DNA mass in each condition. After 24 hours, cells were harvested in modified RIPA buffer or Trizol (Invitrogen). Western analyses to detect V5-tagged Dll-1 protein were performed using an HRP-conjugated V5 antibody diluted 1:1500 (Invitrogen).

### Human ES Cell Culture

The human ES cell line, H9 (WiCell), was maintained on mouse embryonic feeder cells in proliferation medium consisting of Knockout DMEM (GIBCO) supplemented with 20% Knockout serum replacement (GIBCO), pyruvate, glutamine, β-mercaptoethanol and human basic fibroblast growth factor. Details of hES cell differentiation and immunostaining can be found in Supplemental Methods.

### Results

miRNA Expression in Mouse ES Cells and ES Cell-Derived Cardiomyocytes

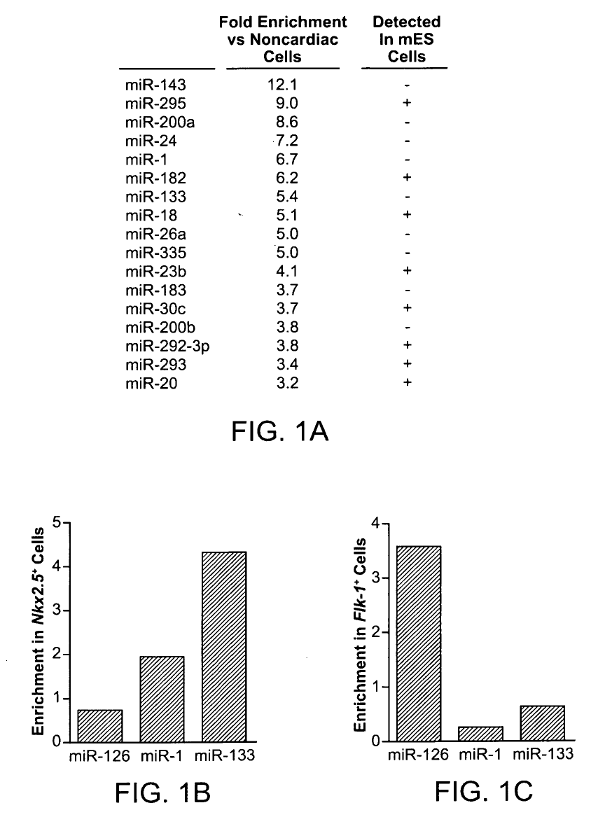

To determine which miRNAs are enriched during differentiation of mouse ES (mES) cells into cardiomyocytes, a mES cell line carrying a green fluorescent protein (GFP) transgene under control of the 13-myosin heavy chain promoter, which is uniquely expressed in differentiated cardiomyocytes, was used. RNA was isolated from GFP⁺ and GFP⁻ cells by fluorescence-activated cell sorting after 13 days of EB differentiation and profiled miRNA expression by microarray analysis. Seventeen miRNAs were enriched at least 3-fold in the GFP⁺ population (FIG. 1a). Approximately half of the miRNAs that were enriched in mES cell-derived cardiomyocytes, including the muscle-specific miRNAs miR-1 and miR-133, were undetectable in undifferentiated mES cells, indicating that they were unique to differentiating cells (FIG. 1a).

To determine whether miR-1 and miR-133 were present and enriched in early cardiac progenitors, a mES cell line carrying a GFP transgene under transcriptional control of a recombinant bacterial artificial chromosome containing the NRx2.5 enhancer was used. This line effectively marks the early emergence of pre-cardiac mesoderm. Sorting of GFP-positive cells in day 4 EBs followed by quantitative RT-PCR (qRT-PCR) revealed that the muscle-specific miRNAs were expressed specifically in the early pre-cardiac mesoderm at this early stage (FIG. 1b), while the vascular endothelium-enriched miRNA, miR-126, was absent (Kuehbacher et al., 2007). Conversely, when vascular progenitors were sorted from day 4 EBs based on their cell surface expression of Flk-1, miR-1 and miR-133 were absent from the Flk-1⁺ mesoderm population in which miR-126 was highly expressed (FIG. 1c). The kinetics of miR-1/miR-133 expression in differentiating whole EBs was also examined. Both miR-1 and miR-133 were detectable as early as day 4 and their levels increased until day 6 after which their relative abundance in the growing EBs diminished other cell types emerged.

FIGS. 1A-C. Identification of miRNAs expressed in ES cell-derived cardiomyocytes. (A) mES cells carrying a GFP transgene under control of the cardiomyocyte-specific β-myosin heavy chain promoter were differentiated for 13 days, sorted by GFP expression, and analyzed by miRNA microarray. miRNAs enriched at least threefold in the GFP⁺ compared to GFP⁻ cell populations are listed along with their fold enrichment and whether they were detected in ES cells. (B, C) qRT-PCR showing enrichment of miR-1 and miR-133 in day 4 NRx2.5-GFP⁺ cardiac progenitors (B) but not in Flk-1⁺ vascular progenitors, which highly express the endothelial-specific miRNA, miR-126 (C).

miR-1 and miR-133 can Promote Mesoderm Differentiation in mES Cells

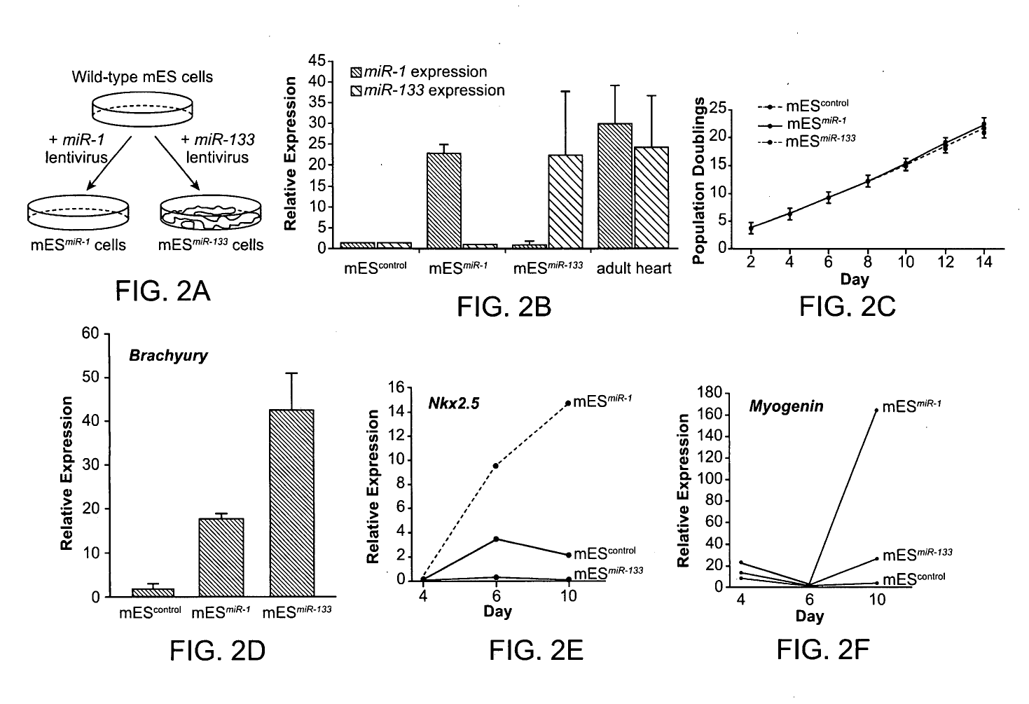

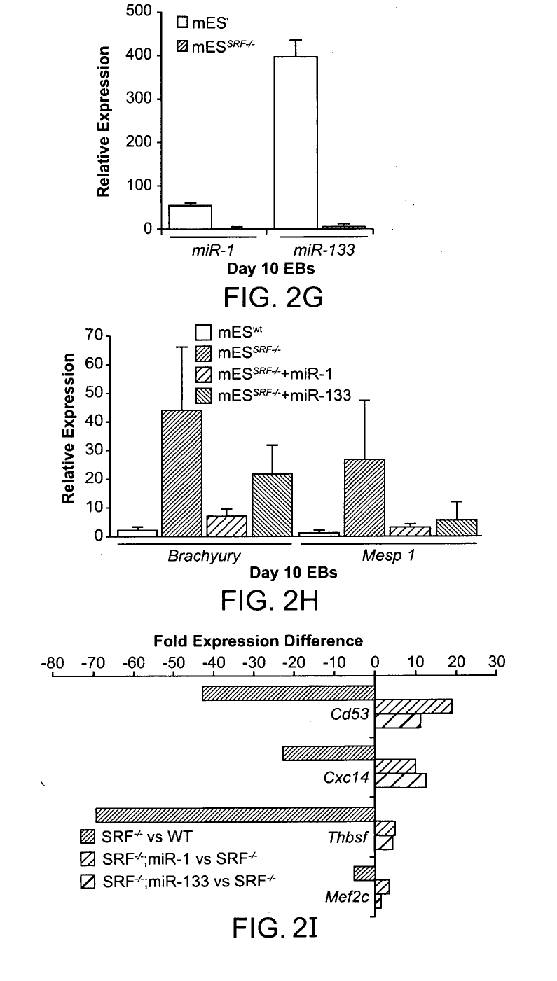

Since miR-1 and miR-133 were not expressed in undifferentiated mES cells, but were specifically enriched in pre-cardiac mesoderm, it was hypothesized that their introduction into mES cells might bias cells toward a muscle lineage. Lentiviruses were used to infect and select ES cell lines expressing miR-1 (mES^{miR-1}) or miR-133 (mES^{miR-133}) (FIG. 2a). The levels of introduced miRNAs approximated those of the endogenous miRNAs in the mouse heart (FIG. 2b). The morphology and doubling time of the cell lines in LIF-containing medium were unaltered (FIG. 2c), and the pluripotency markers Oct-4 and Nanog were expressed at normal levels.

To assess the lineage potential of mES cells expressing miR-1 and miR-133, control, mES^{miR-1}, and mES^{miR-133} cells were differentiated by the hanging drop method. The resulting EBs were collected on days 4, 6, and 10 of differentiation, and the expression of lineage markers was examined by qRT-PCR. Since miR-1 and miR-133 were normally expressed in day 4 pre-cardiac mesoderm, expression of the early mesoderm marker, Brachyury (By), was examined. Bry expression was detected transiently in control EBs at day 4 and then rapidly declined (FIG. 2d). In day 4 EBs expressing miR-1 or miR-133, Bry expression was dramatically enhanced (FIG. 2d), suggesting that both can promote mesodermal gene expression in pluripotent mES cells.

To determine the effects of miR-1 and miR-133 on further differentiation, xpression of NRx2.5, a transcription factor that is one of the earliest cardiac markers, was examined (FIG. 2e). In control EBs, NRx2.5 expression was detected by day 6 and was maintained at day 10. Expression of miR-1 increased NRx2.5 expression at day 6; by day 10, it was ˜7-fold greater than in control EBs. Strikingly, expression of miR-133 blocked induction of NRx2.5 at both time points. A similar expression analysis of Myogenin, an early skeletal muscle marker, was performed to determine the effects of miR-1 and miR-133 on skeletal muscle differentiation. qRT-PCR analysis of Myogenin expression in day 4, 6, or 10 EBs revealed that miR-1, but not miR-133, markedly enhanced Myogenin expression (FIG. 2f).

The increase in NRx2.5 expression, as assessed by qRT-PCR, may represent either an increase in the amount of NRx2.5 expressed per cell or in the number of cells expressing NRx2.5. To distinguish between these two possibilities, the NRx2.5-GFP mES line was infected with control, miR-1-, or miR-133-expressing lentivirus, selected with antibiotic, and differentiated these cells for 10 days. GFP was expressed in more miR-1-expressing EBs, and at higher levels per cell, than in wild-type EBs, and was almost undetectable in miR-133 expressing cells. Thus, miR-1 appears to promote the emergence of both cardiac and skeletal progenitors in mES cells, while miR-133 does not enhance further differentiation of mesoderm precursors into either lineage.

miR-1 or miR-133 Can Rescue Mesoderm Gene Expression in SRF^{-/-} EBs

Efficient methods for stable miRNA knockdown studies in differentiating EBs are not yet available due to the rapid doubling time of ES cells. It was previously shown that expression of the miR-1/miR-133 locus in embryonic mouse hearts is directly dependent on SRF (Zhao et al., 2005). SRF-null ES cells were used as a model for complementation experiments that might reveal the specific contribution of these miRNAs within SRF-null cells (Zhao et al., 2005). It was found that SRF-null EBs failed to activate miR-1 or miR-133 (FIG. 2g), confirming the SRF-dependency in the ES cell system, consistent with in vivo observations. Differentiation of mesodermal progenitors in EBs lacking SRF is weak and delayed (Weinhold et al., 2000). Surprisingly, however, it was found that Bry expression persisted in SRF-null EBs, even after 10 days of differentiation, reflecting delayed or arrested differentiation of mesodermal progenitors that normally downregulate Bry by day 5 (FIG. 2h). Despite the many genes dysregulated in SRF-null EBs, re-introduction of miR-1 in SRF-null ES cells rescued the abnormal accumulation of Bry⁺ progenitors at day 10 of differentiation, with Bry levels returning close to wild-type levels. Introduction of miR-133 had an intermediate effect on the level of Bry expression at day 10, but Bry levels were still significantly elevated. SRF^{-/-} ES cells also displayed elevated expression of Mesp1, a marker of nascent cardiac mesoderm that is usually downregulated as differentiation progresses (Saga et al., 1996) and this was similarly corrected by reintroduction of miR-1 or miR-133 (FIG. 2h). These data suggest miR-1, and to a lesser degree, miR-133, can promote the progression of mesodermal progenitors and that the arrest of mesodermal progenitors in the absence of SRF may be largely due to the absence of this family of miRNAs.

Consistent with the changes in Bry expression, expression of miR-1 or miR-133 restored the expression of a number of mesodermal genes in day 10 SRF-null EBs (FIG. 2i). Blood cell-specific genes, such as Cd53, CxC14, and Thbs1, were dramatically downregulated in SRF^{-/-} EBs, reflecting the loss of hematopoietic lineages in the absence of SRF. However, their expression was reinitiated upon reintroduction of miR-1 or miR-133, likely representing relief of the block to mesodermal differentiation. Even expression of Mef2c, a major regulator of muscle lineages (Li et al., 1997), was restored by miR-1 and, to a lesser extent, by miR-133.

FIGS. 2A-I. Effects of miR-1 and miR-133 on mesoderm differentiation. (A) Schematic of methods used to express miRNAs in mES cells. mES cells were infected with lentiviruses expressing miR-1 or miR-133 under control of a heterologous EF-1 promoter. Stably infected cells were selected based on their resistance to blasticidin in order to generate stable miRNA-expressing mES cell lines (mES^{miR-1} and mES^{miR-133}). (B) qRT-PCR results confirmed the expression of miR-1 and miR-133; expression of the unintroduced miRNA was unchanged. miR-1 and miR-133 were expressed at levels comparable to those in the adult mouse heart. (C) The population doubling times of mES^{miR-1} and mES^{miR-133} cells were similar to those of wild-type mES cells. (D) qRT-PCR analyzing expression of Bry, an early mesoderm marker, in control, mES^{miR-1}, and mES^{miR-133} EBs collected on day 4 of differentiation. Expression of miR-1 or miR-133 increased expression of Bry. (E, F) qRT-PCR analysis of NRx2.5 (E) and Myogenin (F) expression from day 4, 6, or 10 EBs formed from control, mES^{miR-1}, or mES^{miR-133} cells. Control EBs displayed an induction of NRx2.5 expression over time that was enhanced by miR-1 and suppressed by miR-133. Induction of Myogenin expression was enhanced by miR-1, but not by miR-133. (G) Expression of miR-1 and miR-133 was undetectable in day 10 SRF^{-/-} EBs by qRT-PCR. (H) Overexpression of miR-1 and to a lesser extent, miR-133, in SRF^{-/-} EBs restored the Bry and Mesp1 downregulation typical of wild-type cells. (I) Expression of Cd53, Cxc14, and Thbs1, which mark hematopoietic lineages, and of Mef2c, which encodes a major regulator of muscle differentiation, was partially rescued in SRF^{-/-} EBs upon expression of miR-1 or miR-133.

miR-1 and miR-133 Suppress Endoderm Differentiation in mES Cells

It has been proposed that in some contexts miRNAs function in a “fail-safe” mechanism to clear latent gene expression by targeting pathways that should not be activated in a particular cell type (Hornstein et al., 2005). It was investigated whether miR-1 and miR-133 might not only promote muscle lineage decisions, but also reinforce them by repressing nonmuscle gene expression. First, control, mES^{miR-1}, and mES^{miR-133} ES cells were differentiated in the presence of recombinant nodal, a potent inducer of endoderm differentiation in mES cells (Vallier et al., 2004; Pfendler et al., 2005). As expected, nodal stimulated expression of the endoderm markers α-Fetoprotein (Afp) and Hnf4α control EBs (FIG. 3a,b). These markers were expressed at dramatically lower levels in mES^{miR-1} and mES^{miR-133} EBs than in control EBs, indicating that miR-1 or miR-133 can each function as potent repressors of endoderm gene expression during differentiation of pluripotent mES cells (FIG. 3a,b).

miR-1 and miR-133 Suppress Neural Differentiation From mES Cells

Next, it was asked whether miR-1 or miR-133 could also suppress neuroectoderm gene expression from pluripotent mES cells. Control, mES^{miR-1}, and mES^{miR-133} ES cells were differentiated in the presence of retinoic acid (RA), a potent inducer of neural differentiation (Bain et al., 1995; Bain et al., 1996). RA-treated, control EBs expressed high levels of neural cell adhesion molecule 1 (Ncam1), a marker of mature neurons, by day 10 of differentiation, but Ncam1 induction was suppressed in both mES^{miR-1} and mES^{miR-133} EBs (FIG. 3c). Expression of Nestin, which is restricted largely to neural progenitor cells and is downregulated upon further neural differentiation (Hockfield and McKay, 1985), was also examined. Nestin expression persisted beyond day 10 in mES^{miR-1} and mES^{miR-133} EBs, well after its decline in control EBs, suggesting an accumulation of neural progenitors (FIG. 3d). Suppression of endoderm or neuroectoderm differentiation was not observed when an endothelial-enriched microRNA, miR-126, was similarly introduced into mES cells, indicating specificity of miR-1 and miR-133 effects. These data indicate that both miR-1 and miR-133 can curtail the differentiation of pluripotent cells into mature neurons, even as cells are pushed toward that lineage by timed administration of RA.

Coordinate Dysregulation of Gene Expression in mES^{miR-1} and mES^{miR-133} EBs

To more broadly assess the influence of miR-1 or miR-133 on lineage specification and gene expression, mRNA expression microarray analyses were performed on day 10 control, mES^{miR-1}, and mES^{miR-133} EBs. Consistent with the similar effects of miR-1 and miR-133 on repression of nonmuscle gene expression, the vast majority of genes were coordinately regulated between mES^{miR-1} and mES^{miR-133} EBs (FIG. 3e). Among the most highly downregulated genes in both the mES^{miR-1} and mES^{miR-133} EBs were the early endoderm markers, Afp and Hnf4α, consistent with the qRT-PCR results from EBs treated with nodal (FIG. 3f). Expression of other genes normally enriched in endodermal structures, such as those encoding apolipoproteins, was also downregulated in both mES^{miR-1} and mES^{miR-133} EBs (FIG. 3f). These results support the idea that miR-1 and miR-133 can suppress endoderm specification and differentiation.

Among the most highly upregulated genes in both mES^{miR-1} and mES^{miR-33} EBs were those associated with neuroectoderm specification and early neural differentiation These included the early neurogenic transcription factors, Neurod4, Phox2b, and Myt1 and a number of Hox genes involved in neural specification (FIG. 3f). This is consistent with the observation of persistent Nestin expression in mES^{miR-1} and mES^{miR-133}-derived EBs and the apparent disruption of late-stage neuronal differentiation by these miRNAs.

A number of mesodermal genes were also commonly dysregulated in both mES^{miR-1} and mES^{miR-133} EBs (FIG. 3f). Runx2 and Twist1, which are highly expressed in developing bone (Ducy et al., 1997; Bialek et al., 2004), were both upregulated, further supporting the conclusion that mesoderm specification is increased in miR-1- or miR-133-expressing EBs. However, a number of genes encoding sarcomeric proteins found in differentiated muscle cells were decreased in both mES^{miR-1} and mES^{miR-133} EBs. The mechanism for diminished sarcomeric gene expression in EBs may differ in the two cells lines: mesodermal progenitors in the mES^{miR-133} EBs likely fail to differentiate into muscle, remaining in the progenitor state, while differentiating muscle cells in mES^{miR-1} EBs may prematurely exit the cell cycle resulting in fewer cardiac cells, as was observed upon overexpression of miR-1 in the mouse heart (Zhao et al., 2005). Both would result in underrepresented muscle gene expression and each is consistent with the current understanding of miR-1 and miR-133 function.

FIGS. 3A-F. Both miR-1 and miR-133 suppress endoderm and neuroectoderm differentiation in mES cells. (A, B) qRT-PCR analysis of the endoderm markers Afp (A) or Hnf4α(B) from day 4, 6, or 10 nodal-treated EBs formed from control, mES^{miR-1} or mES^{miR-133} cells. Induction of Afp and Hnf4α expression normally observed during differentiation in the presence of nodal was suppressed by expression of miR-1 or miR-133. (C) qRT-PCR analysis of the neural marker Ncam1 from day 4, 6, or 10 RA-treated EBs formed from control, mES^{miR-1} or mES^{miR-133} cells. Expression of miR-1 or miR-133 suppressed the induction of Ncam normally observed during differentiation in the presence of RA. (D) qRT-PCR analysis of the neural progenitor marker Nestin in day 4, 8, or 10 RA-treated EBs formed from control, mES^{miR-1} or mES^{miR-133} cells. Nestin expression declined in wild-type EBs by day 10 as neurons differentiated, but was maintained in mES^{miR-1} and mES^{miR-133} EBs. (E) Plot comparing results from mRNA expression microarray analyses of day 10 control, mES^{miR-1}, and mES^{miR-133} EBs. Plot shows that most genes were coordinately regulated. (F) Examples of genes that were coordinately regulated in mES^{miR-1} and mES^{miR-133} EBs compared to controls.

miR-1 and miR-133 Suppress Neural Differentiation during Teratoma Formation

To examine the ability of miR-1 and miR-133 to suppress nonmesodermal lineages in a more in vivo setting, wild-type or miRNA-expressing mES cells were injected subcutaneously into SCID mice and monitored their differentiation in vivo. Transplanted cells of each line formed teratomas in the recipients and were analyzed 6 weeks after inoculation. Teratomas from control, mES^{miR-1}, or mES^{miR-133} cells included derivatives of all three embryonic germ layers, but the control teratomas were much more homogeneous. As shown by immunostaining with βIII-tubulin antibodies, teratomas from control mES cells were composed mostly of differentiated neurons. In contrast, teratomas formed from mES^{miR-1} or mES^{miR-133} cells had far fewer differentiated neuronal cells.

Based on the analyses of neural differentiation in EBs, immunostained teratomas were also immunostained using an antibody to nestin. Control teratomas were fully differentiated and contained only rare pockets of nestin-positive neural progenitors, as expected. However, mES^{miR-1} and mES^{miR-133} teratomas contained abundant nestin-positive cells even after 6 weeks of development, suggesting an arrest of neural differentiation at the progenitor stage. The accumulation of nestin-positive progenitors in these teratomas further supports the idea that miR-1 and miR-133 permit specification of the ectodermal lineage from pluripotent mES cells, but inhibit complete differentiation of neural progenitor cells into neurons.

Teratomas were also immunostained using an antibody to smooth muscle α-actin, a marker of smooth muscle and immature striated muscle cells (cardiac and skeletal). Consistent with the promesodermal effects of miR-1 and miR-133 in EBs, teratomas derived from mES^{miR-1} and mES^{miR-133}-derived teratomas had more cells on average expressing smooth muscle α-actin than control. High magnification views of immunostained sections demonstrated the specificity of each antibody.

The Notch Ligand, Delta-Like 1, is Translationally Repressed by miR-1

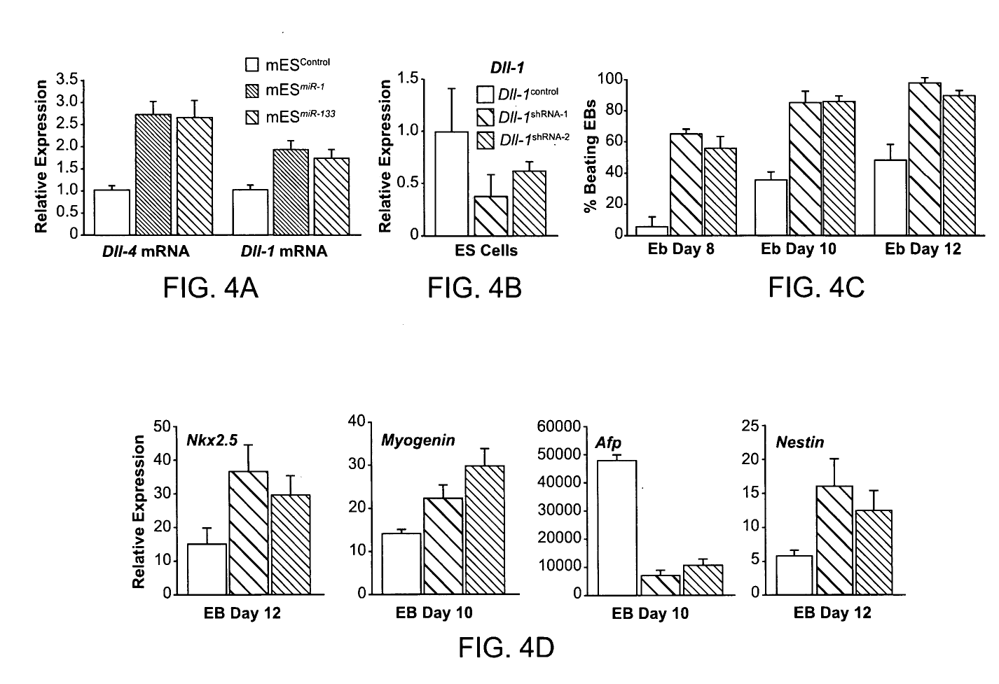

miRNAs likely function by regulating numerous pathways, but in some cases a subset serve as the “major” effectors. Notch signaling can promote neural differentiation and inhibit muscle differentiation in ES cells (Nemir et al., 2006; Lowell at al., 2006), which is opposite of miR-1's effects. It was hypothesized that miR-1-mediated repression of Notch signaling may contribute to the observed effects of miR-1 in mES cells. It had previously been shown that miR-1 directly targets the Notch ligand delta in Drosophila for repression (Kwon et al., 2005). Three orthologs of Drosophila delta have been identified in mice-Dll-1, Dll-3, and Dll-4. Dll-1 and Dll-4, but not Dll-3, contained putative miR-1 or miR-133 binding sites in their 3′ UTR. As shown by qRT-PCR analysis, mRNA expression of Dll-1 and Dll-4 was similar in mES^{miR-1} and mES^{miR-133} cells and somewhat higher than in control mES cells (FIG. 4a).

Since miRNAs can block the translation of target mRNAs, Dll-1 and Dll-4 protein levels were examined in all three mES cell lines. mES^{miR-1}, mES^{miR-133}, and control cells had similar levels of Dll-4 by immunocytochemistry and Western analysis. Quantitative analysis of endogenous Dll-1 protein was not possible due to the lack of published Dll-1 antibodies that function in Western blots. However, mES^{miR-1} cells had consistently decreased Dll-1 protein levels by immunocytochemistry despite having normal levels of Dll-1 mRNA, consistent with translational inhibition of Dll-1 by miR-1. Although a potential miR-1 binding site in the Dll-1 3′-UTR has extensive, conserved sequence matching and is present in an accessible region with little secondary structure, repression through this site was not transferable to the luciferase 3′-UTR in the surrogate assay commonly employed to test specific binding sites. However, miR-1 potently repressed protein, but not mRNA expression of an epitope-tagged Dll-1 containing the full 3′UTR in a dose-dependent manner indicating translational inhibition of Dll-1 in mammalian cells.

Dll-1 Knockdown in mES Cells Partially Recapitulates miR-1 Activity

To determine whether downregulation of Dll-1 protein by miR-1 could account for a subset of the effects of miR-1 on cell lineage decisions, short hairpin RNA (shRNA) constructs directed against distinct regions of Dll-1 were used to generate two different Dll-1^{shRNA} cell lines (Dll-1^{shRNA-1} and Dll-1^{shRNA-2}). The Dll-1 mRNA level was about 62% lower in Dll-1^{shRNA-1} cells and 40% lower in Dll-1^{shRNA-2} cells than in a control line expressing a scrambled shRNA construct, as shown in FIG. 4B. Oct3/4 levels and cell morphology were unaltered. EBs formed from Dll-1^{shRNA} cells had a much greater propensity toward cardiomyocyte differentiation and formed beating cardiomyocytes earlier than control EBs, as shown in FIG. 4C. By day 12 of differentiation, 89% of EBs formed from Dll-1^{shRNA-1} cells and 97% of EBs from Dll-1^{shRNA-2} cells contained beating cardiomyocytes compared to 48% of Dll-1^{control} EBs. NRx2.5 expression, marking cardiac progenitors, was also more highly induced in Dll-1^{shRNA} than in control EBs, as were NRx2.5-GFP-positive cells, as shown in FIG. 4E. In addition, Myogenin expression was higher in Dll-1^{shRNA} EBs compared to controls, as shown in FIG. 4D. Although the effect of Dll-1 knockdown on NRx2.5 and myogenin expression was not as robust as miR-1 expression, the trends were similar. These results indicate that depletion of Dll-1 increases muscle differentiation from mES cells and suggest that miR-1 may promote cardiac differentiation, in part, by downregulating Dll-1 protein.

qRT-PCR analyses were also performed on EBs formed from Dll-1^{shRNA} cell lines to determine if suppression of ectodermal and endodermal lineages by miR-1 might also involve Dll-1 downregulation. Expression of the endoderm markers Afp (FIG. 4D) and Hnf40α was lower in Dll-1^{shRNA} EBs than in Dll-1^{control} EBs. Moreover, expression of Nestin, which decreased between days 10 and 12 as neurons differentiated in Dll-1^{control} EBs, was increased during this period in both lines of Dll-1^{shRNA} EBs (FIG. 4D). Thus, loss of Dll-1 also represses endoderm differentiation and results in persistence of neural progenitor gene expression.

FIGS. 4A-D. Dll-1 protein levels are negatively regulated by miR-1 in mES cells, and knockdown of Dll-1 expression recapitulates many effects of miR-1 expression. (A) Dll-1 and Dll-4 mRNA levels, assessed by qRT-PCR, were somewhat higher in mES^{miR-1} and mES^{miR-133} cells than in controls. (B) Dll-1 mRNA levels, assessed by qRT-PCR, were 62% and 40% lower in the Dll-1^{shRNA-1} and Dll-1^{shRNA-2} cell lines, respectively, than in the control line. (C) EBs formed from Dll-1^{control}, Dll-1^{shRNA-1} and Dll-1^{shRNA-2} ES cells were scored for beating cardiomyocytes on days 8, 10, and 12 of differentiation. Beating cardiomyocytes appeared earlier and were more numerous in EBs from Dll-1^{shRNA} cell line than in EBs from the control line. (D) qRT-PCR analyses of NRx2.5, Myogenin, Afp, and Nestin expression in EBs generated from Dll-1^{control}, Dll-1^{shRNA-1} and Dll-1^{shRNA-2} ES cells. Knocking down Dll-1 recapitulated the increased Myogenin expression, decreased Afp expression and sustained Nestin expression observed upon expression of miR-1. The designations for the Dll-1^{control}, Dll-1^{snRNA-1} and Dll-1^{shRN-2} bars are the same for FIGS. 4B, 4C, and 4D.

Effects of miR-1 or miR-133 in Human ES Cells

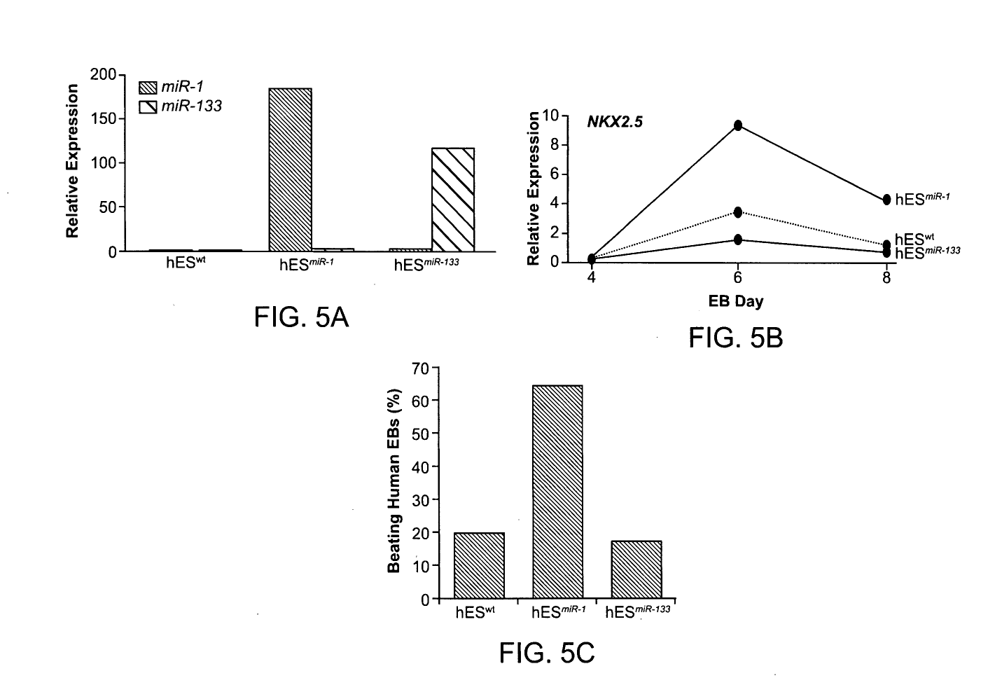

Human ES (hES) cells often behave differently than mES cells. To investigate whether miR-1 or miR-133 function similarly in the two cell types, the H9 hES cell line was infected with the same lentiviruses encoding either miR-1 or miR-133. Expression was verified by qRT-PCR (FIG. 5a). The resulting hES^{miR-1} and hES^{miR-133} cell lines were differentiated as EBs in suspension and collected on days 4, 6, and 8. NKX2.5 expression was detectable by qRT-PCR in control human EBs by day 6 and decreased overall by day 8 (FIG. 5b). As in the mouse EBs, hES^{miR-1} EBs had higher levels of NKX2.5 expression than controls, while hES^{miR-133} EBs failed to induce NKX2.5 expression to the levels observed in controls (FIG. 5b). Consistent with this, it was also found that the percentage of hES miR-1 EBs with beating cardiac cells on day 18 of differentiation was more than 3-fold higher than in wild-type EBs, while hES miR-133 EBs did not display enhanced cardiomyocyte formation (FIG. 5c). Thus, regulation of cardiac differentiation by miR-1 and miR-133 appears to be grossly similar in hES and mES cells.

To examine the effects of miR-1 or miR-133 expression on neuroectoderm differentiation in hES cells, day 18 control, hES^{miR-1}, and hES^{miR-133} EBs were immunostained with antibodies recognizing nestin or βIII-tubulin. Like miRNA-expressing mouse EBs, hES^{miR-1} and hES^{miR-}133 EBs accumulated more nestin-positive progenitors than control human EBs. As in the mouse ES cells studies, there were fewer βIII-tubulin positive neural cells in hES miR-133 EBs compared to controls, although this effect was not consistent for hES^{miR-}1 cells. These results demonstrate that the muscle-specific miRNAs miR-1 and miR-133 have similar, but somewhat unique effects on the differentiation of hES and mES cells, and suggest that miRNAs may be useful for coaxing and repressing differentiation of human or mouse ES cells into particular lineages.

FIGS. 5A-C. Effects of miR-1 or miR-133 expression in hES cells. (A) Lentivirus-mediated expression of miR-1 or miR-133 in hES cells was verified by qRT-PCR. (B) NKX2.5 expression assessed by qRT-PCR in hEBs collected on days 4, 6, and 8. Overexpression of miR-1 in hES cells increased NKX2.5 expression compared to wild type, while miR-133 expression led to decreased NKX2.5 induction. (C) Human EBs were scored for beating on day 18 of differentiation. Expression of miR-1 increased the number of beating human EBs, while expression of miR-133 did not.

### REFERENCES

Ambros, V. (2004). The functions of animal microRNAs. Nature 431, 350-355.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat. Genet. 25, 25-29.
Bain, G., Kitchens, D., Yao, M., Huettner, J. E., and Gottlieb, D. I. (1995). Embryonic stem cells express neuronal properties in vitro. Dev. Biol. 168, 342-357.
Bain, G., Ray, W. J., Yao, M., and Gottlieb, D. I. (1996). Retinoic acid promotes neural and represses mesodermal gene expression in mouse embryonic stem cells in culture. Biochem. Biophys. Res. Commun. 223, 691-694.
Bialek, P., Kern, B., Yang, X., Schrock, M., Sosic, D., Hong, N., Wu, H., Yu, K., Ornitz, D. M., Olson, E. N., Justice, M. J., Karsenty, G. (2004). A twist code determines the onset of osteoblast differentiation. Dev. Cell 6, 423-435.
Chen, J. F., Mandel, E. M., Thomson, J. M., Wu, Q., Callis, T. E., Hammond, S. M., Conlon, F. L., and Wang, D. Z. (2006). The role of microRNA-1 and microRNA-133 in skeletal muscle proliferation and differentiation. Nat. Genet. 38, 228-233.
Ducy, P., Zhang, R., Geoffroy, V., Ridall, A. L., Karsenty, G. (1997). Osf2/Cbfa1: a transcriptional activator of osteoblast differentiation. Cell 89, 747-754.
Dudoit, S., Gentleman, R. C., and Quackenbush, J. (2003). Open source software for the analysis of microarray data. Biotechniques (Suppl), 45-51.
He, L., and Hannon, G. J. (2004). MicroRNAs: small RNAs with a big role in gene regulation. Nat. Rev. Genet. 5, 522-531.
Hockfield, S., and McKay, R. D. (1985). Identification of major cell classes in the developing mammalian nervous system. J. Neurosci. 5, 3310-3328.
Hornstein, E., Mansfield, J. H., Yekta, S., Hu, J. K., Harfe, B. D., McManus, M. T., Baskerville, S., Bartel, D. P., Tabin, C. J. (2005). The microRNA miR-196 acts upstream of Hoxb8 and Shh in limb development. Nature 438, 671-674.
Houbaviy, H. B., Murray, M. F., and Sharp, P. A. (2003). Embryonic stem cell-specific MicroRNAs. Dev. Cell 5, 351-35
Kanellopoulou, C., Muljo, S. A., Kung, A. L., Ganesan, S., Drapkin, R., Jenuwein, T., Livingston, D. M., and Rajewsky, K. (2005). Dicer-deficient mouse embryonic stem cells are defective in differentiation and centromeric silencing. Genes Dev. 19, 489-501.
Kuehbacher, A., Urbich, C., Zeiher, A. M., Dimmeler, S. (2007). Role of Dicer and Drosha for endothelial microRNA expression and angiogenesis. Circ. Res. 101, 59-68.
Kwon, C., Han, Z., Olson, E. N., and Srivastava, D. (2005). MicroRNA1 influences cardiac differentiation in Drosophila and regulates Notch signaling. Proc. Natl. Acad. Sci. USA 102, 18986-18991.
Kwon, C., Arnold, J., Hsiao, E. C., Taketo, M. M., Conklin, B. R., and Srivastava, D. (2007). Canonical Wnt signaling is a positive regulator of mammalian cardiac progenitors. Proc Natl Acad Sci USA 104, 10894-10899.
Li, H., Capetanaki, Y. (1997). AN E box in the desmin promoter cooperates with the E box and Mef-2 sites of a distal enhancer to direct muscle-specific transcription. EMBO J. 13, 3580-3589.
Loebel, D. A., Watson, C. M., De Young, R. A., and Tam, P. P. (2003). Lineage choice and differentiation in mouse embryos and embryonic stem cells. Dev. Biol. 264, 1-14.
Lowell, S., Benchoua, A., Heavey, B., and Smith, A. G. (2006). Notch promotes neural lineage entry by pluripotent embryonic stem cells. PLoS Biol 4, e121.
Maltsev, V. A., Wobus, A. M., Rohwedel, J., Bader, M., and Hescheler, J. (1994). Cardiomyocytes differentiated in vitro from embryonic stem cells developmentally express cardiac-specific genes and ionic currents. Circ. Res. 75, 233-244.
Murchison, E. P., Partridge, J. F., Tam, O. H., Cheloufi, S., and Hannon, G. J. (2005). Characterization of Dicer-deficient murine embryonic stem cells. Proc. Natl. Acad. Sci. USA 102, 12135-12140.
Nemir, M., Croquelois, A., Pedrazzini, T., and Radtke, F. (2006). Induction of cardiogenesis in embryonic stem cells via downregulation of Notch1 signaling. Circ. Res. 98, 1471-1478.
Pfendler, K. C., Catuar, C. S., Meneses, J. J., and Pedersen, R. A. (2005). Overexpression of Nodal promotes differentiation of mouse embryonic stem cells into mesoderm and endoderm at the expense of neuroectoderm formation. Stem Cells Dev. 14, 162-172.
Rao, P. K., Kumar, R. M., Farkhondeh, M., Baskerville, S., and Lodish, H. F. (2006). Myogenic factors that regulate expression of muscle-specific microRNAs. Proc. Natl. Acad. Sci. USA 103, 8721-8726.
Saga, Y., Hata, N., Kobayashi, S., Magnuson, T., Seldin, M. F., Taketo, M. M. (1996).
Mesp1: a novel basic helix-loop-helix protein expressed in the nascent mesodermal cells during mouse gastrulation. Development 122, 2769-2778.
Sokol, N. S., and Ambros, V. (2005). Mesodermally expressed Drosophila microRNA-1 is regulated by Twist and is required in muscles during larval growth. Genes Dev 19, 2343-2354.
Srivastava, D., Thomas, T., Lin, Q., Kirby, M. L., Brown, D., and Olson, E. N. (1997). Regulation of cardiac mesodermal and neural crest development by the bHLH transcription factor, dHAND. Nat Genet. 16, 154-160.
Srivastava, D. (2006). Making or breaking the heart: from lineage determination to morphogenesis. Cell 126, 1037-1048.
Vallier, L., Reynolds, D., and Pedersen, R. A. (2004). Nodal inhibits differentiation of human embryonic stem cells along the neuroectodermal default pathway. Dev. Biol. 275, 403-421.
Wang, Y., Medvid, R., Melton, C., Jaenisch, R., and Blelloch, R. (2007). DGCR8 is essential for microRNA biogenesis and silencing of embryonic stem cell self-renewal. Nat. Genet. 39, 380-385.
Weinhold, B., Schratt, G., Arsenian, S., Berger, J., Kamino, K., Schwarz, H., Ruther, U., and Nordheim, A. (2000). Srf(−/−) ES cells display non-cell-autonomous impairment in mesodermal differentiation. EMBO J. 19, 5835-5844.
Wu (2004). Wu, Z., and Irizarry, R. A. (2004). Preprocessing of oligonucleotide array data. Nat Biotechnol 22, 656-658; author reply 658
Yamagishi, H., Yamagishi, C., Nakagawa, O., Harvey, R. P., Olson, E. N., and Srivastava, D. (2001). The combinatorial activities of NRx2.5 and dHAND are essential for cardiac ventricle formation. Dev Biol 239, 190-203.
Zhao, Y., Samal, E., and Srivastava, D. (2005). Serum response factor regulates a muscle-specific microRNA that targets Hand2 during cardiogenesis. Nature 436, 214-220.
Zhao, Y., and Srivastava, D. (2007). A developmental view of microRNA function. Trends Biochem Sci 32, 189-197.
Zhao, Y., Ransom, J. F., Li, A., Vedantham, V., von Drehle, M., Muth, A. N., Tsuchihashi, T., McManus, M. T., Schwartz, R. J., and Srivastava, D. (2007). Dysregulation of cardiogenesis, cardiac conduction, and cell cycle in mice lacking miRNA-1-2. Cell 129, 303-317.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of inducing cardiomyogenesis in a stem cell or progenitor cell, the method comprising introducing into a stem cell or a progenitor cell a microRNA-1 (miR-1) nucleic acid or a nucleic acid comprising a nucleotide sequence encoding a miR-1 nucleic acid, thereby generating a cardiomyocyte.

2. The method of claim 1, wherein the stem cell is an embryonic stem cell.

3. The method of claim 1, wherein the stem cell is an induced pluripotent stem cell.

4. The method of claim 1, wherein the miR-1 nucleic acid comprises a stem-loop forming nucleotide sequence.

5. The method of claim 4, wherein the miR-1 nucleic acid comprises a nucleotide sequence having at least about 75% nucleotide sequence identity to nucleotides 7-69 of the nucleotide sequence set forth in SEQ ID NO:1.

6. The method of claim 4, wherein the miR-1 nucleic acid comprises a nucleotide sequence having at least about 85% nucleotide sequence identity to nucleotides 7-69 of the nucleotide sequence set forth in SEQ ID NO:1.

7. The method of claim 1, wherein the miR-1 nucleic acid comprises a mature miR-1 nucleotide sequence.

8. The method of claim 7, wherein the miR-1 nucleic acid comprises the nucleotide sequence set forth in SEQ ID NO:2.

9. The method of claim 1, wherein the nucleic acid encoding a miR-1 nucleic acid is an expression construct, and wherein the miR-1-encoding nucleotide sequence is operably linked to a transcription regulatory element.

10. The method of claim 9, wherein the transcription regulatory element is a constitutive promoter functional in the stem or progenitor cell.

11. The method of claim 9, wherein the transcription regulatory element is an inducible promoter.

12. The method of claim 1, further comprising introducing a miR-133 nucleic acid, or a nucleic acid comprising a nucleotide sequence encoding a miR-133 nucleic acid, into the stem or progenitor cell.

13. The method of claim 1, wherein the stem or progenitor cell is present in a matrix.

14. The method of claim 1, further comprising isolating the cardiomyocyte.

15. The method of claim 14, further comprising associating the cardiomyocyte with a matrix.

16. A method of inducing expansion of a cardiac progenitor cell, the method comprising introducing into a cardiac progenitor cell a microRNA-133 (miR-133) nucleic acid, or a nucleic acid comprising a miR-133 nucleic acid.

17. The method of claim 16, wherein the miR-133 nucleic acid comprises a stem-loop forming nucleotide sequence.

18. The method of claim 17, wherein the miR-133 nucleic acid comprises a nucleotide sequence having at least 75% nucleotide sequence identity with nucleotides 7-83 of the nucleotide sequence set forth in SEQ ID NO:5.

19. The method of claim 17, wherein the miR-133 nucleic acid comprises a nucleotide sequence having at least 85% nucleotide sequence identity with nucleotides 7-83 of the nucleotide sequence set forth in SEQ ID NO:5.

20. The method of claim 16, wherein the miR-133 nucleic acid comprises a mature miR-133 nucleotide sequence.

21. The method of claim 20, wherein the miR-133 nucleic acid comprises the nucleotide sequence set forth in SEQ ID NO:8.

22. A genetically modified stem cell or progenitor cell, or a progeny thereof, wherein the genetically modified stem cell or progenitor cell comprises an exogenous nucleic acid selected from an exogenous miR-1 nucleic acid, an exogenous miR-133 nucleic acid, an exogenous nucleic acid comprising a nucleotide sequence encoding a miR-1 nucleic acid, and an exogenous nucleic acid comprising a nucleotide sequence encoding a miR-133 nucleic acid.

23. The genetically modified stem cell or progenitor cell of claim 22, wherein the stem cell is an induced pluripotent stem cell.

24. The genetically modified stem cell or progenitor cell of claim 22, wherein the exogenous nucleic acid is a recombinant expression construct.

25. The genetically modified stem cell or progenitor cell of claim 22, wherein the exogenous nucleic acid is stably integrated into the genome of the cell.

26. The genetically modified stem cell of claim 25, wherein the exogenous nucleic acid is a recombinant lentivirus construct.

27. A cardiomyocyte derived from the genetically modified stem cell or progenitor cell of claim 22.

28. A composition comprising a genetically modified stem cell or progenitor cell of claim 22.

29. The composition of claim 28, wherein the composition comprises a matrix component.

30. The composition of claim 29, wherein the matrix comprises one or more of collagen, gelatin, fibrin, fibrinogen, laminin, a glycosaminoglycan, elastin, hyaluronic acid, proteoglycan, a glycan, poly(lactic acid), poly(vinyl alcohol), poly(vinyl pyrrolidone), poly(ethylene oxide), cellulose; a cellulose derivative, starch, a starch derivative, poly(caprolactone), and poly(hydroxy butyric acid).

31. The composition of claim 30, further comprising one or more of a growth factor, an antioxidant, a nutritional transporter, and a polyamine.


SEQUENCE LISTING (12 sequences)

SEQ ID NO: 1 - RNA, 71 nt, Homo sapiens
ugggaaacauacuucuuuauaugcccauauggaccugcuaagcuauggaauguaaagaaguauguaucuca

SEQ ID NO: 2 - RNA, 22 nt, Homo sapiens
uggaauguaaagaaguauguau

SEQ ID NO: 3 - RNA, 85 nt, Homo sapiens
accuacucagaguacauacuucuuuauguacccauaugaacauacaaugcuauggaauguaaagaaguauguauuuuugguaggc

SEQ ID NO: 4 - RNA, 72 nt, rat
ucagagcacauacuucuuuauguacccauaugaacauagaaugcuauggaauguaaagaaguguguauuuug

SEQ ID NO: 5 - RNA, 88 nt, Homo sapiens
acaaugcuuugcuagagcugguaaaauggaaccaaaucgccucuucaauggauuugguccccuucaaccagcuguagcuaugcauuga

SEQ ID NO: 6 - RNA, 102 nt, Homo sapiens
gggagccaaaugcuuugcuagagcugguaaaauggaaccaaaucgacuguccaauggauuugguccccuucaaccagcuguagcugugcauugauggcgccg

SEQ ID NO: 7 - RNA, 119 nt, Homo sapiens
ccucagaagaaagaugcccccugcucuggcuggucaaacggaaccaaguccgucuuccugagagguuugguccccuucaaccagcuacagcagggcuggcaaugcccaguccuuggaga

SEQ ID NO: 8 - RNA, 22 nt, Homo sapiens
uuugguccccuucaaccagcug

SEQ ID NO: 9 - DNA, 3366 bp, Homo sapiens
cgtgggatttccagaccgcggctttctaatcggctcgggaggaagctctgcagctctcttgggaattaagctcaatctctggactctctctctttctctttctccccctccctctcctgcgaagaagctcaagacaaaaccaggaagccggcgaccctcacctcctcgggggctgggaggaaggaggaaaacgaaagtcgccgccgccgcgctgtcccccgagagctgcctttcctcgggcatccctggggctgccgcgggacctcgcagggcggatataaagaaccgcggccttgggaagaggcggagaccggcttttaaagaaagaagtcctgggtcctgcggtctggggcgaggcaagggcgcttttctgcccacgctccccgtggcccatcgatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccaccgctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaatgacaaaaaaaaaaaaaaaaaaaaaaaaaaaaaaaaaaaaaaaaaaaaaaaaaaaaaaaaaa

SEQ ID NO: 10 - RNA, 68 nt, mouse
gcuaaagcugguaaaauggaaccaaaucgccucuucaauggauuugguccccuucaaccagcuguagc

SEQ ID NO: 11 - RNA, 104 nt, mouse
agaagccaaaugcuuugcugaagcugguaaaauggaaccaaaucagcuguuggauggauuugguccccuucaaccagcuguagcugcgcauugaucacgccgca

SEQ ID NO: 12 - RNA, 119 nt, mouse
ccuccaaagggaguggcccccugcucuggcuggucaaacggaaccaaguccgucuuccugagagguuugguccccuucaaccagcuacagcagggcuggcaaagcucaauauuuggaga